(19) United States
(12) Patent Application Publication
(10) Pub. No.: US 2026/0130861 A1
(43) Pub. Date: May 14, 2026

(54) THERAPEUTIC NANOPARTICLES WITH ANTIOXIDANT AND IRON-CHELATION ACTIVITY

(71) Applicant: University of Oregon, Eugene, OR (US)

(72) Inventors: Danielle Benoit, Eugene, OR (US); Cameron J. Moore, Eugene, OR (US)

(73) Assignee: University of Oregon, Eugene, OR (US)

(21) Appl. No.: 19/390,290

(22) Filed: Nov. 14, 2025

Related U.S. Application Data
(60) Provisional application No. 63/720,621, filed on Nov. 14, 2024.

(51) Int. Cl. A61K 9/51; A61P 39/04; B82Y 5/00

(52) U.S. Cl. CPC A61K 9/5138; A61K 9/5192; A61P 39/04; B82Y 5/00

(57) ABSTRACT

Therapeutic nanoparticles with antioxidant and/or iron-chelation activity comprise a plurality of polymers. In some aspects, the polymers have structures according to Formula I:

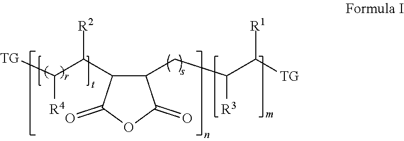

where m, n, and t, independently are integers ≥1; each TG independently is a terminating group; each R¹, R², R³, and R⁴, independently, for each occurrence, is H or comprises an aryl group; each r independently, for each occurrence, is 1 or 0; and each s independently, for each occurrence, is 1 or 0; and provided that, (i) at least one R¹, R², R³, or R⁴, is not, or is other than, phenyl, (ii) one of R¹ or R³, for each occurrence, comprises the aryl group, and (iii) one of R² or R⁴, for each occurrence, comprises the aryl group. The nanoparticles may be useful for modulating inflammation and/or iron-dependent cell death.

### CROSS REFERENCE TO RELATED APPLICATIONS

This application claims the benefit of the earlier filing date of U.S. Provisional Application No. 63/720,621, filed Nov. 14, 2024, which is incorporated by reference in its entirety herein.

### FIELD

Therapeutic nanoparticles with antioxidant and iron-chelation activity are disclosed, along with methods of making and using the therapeutic nanoparticles.

### BACKGROUND

As mammalian cells age, dysregulated iron metabolism results in excess iron accumulation, oxidative damage, and a chronic inflammatory state termed “inflammaging”. This leads to ferroptosis, a unique form of iron-dependent inflammatory cell death that occurs when antioxidant pathways are depleted by the iron-catalyzed generation of reactive oxygen species (ROS) and phospholipid peroxidation. Since its discovery in 2012, ferroptosis has been implicated in a myriad of age-related inflammatory orthopedic, cardiovascular, and neurodegenerative diseases. For example, growing evidence suggests that age-related impaired fracture healing is a function of increased production of inflammatory cytokines and ROS. Antioxidant and iron chelating therapeutics capable of scavenging ROS and cytosolic iron at fractures offer a promising avenue for reversing the proinflammatory milieu responsible for age-related osteogenic stem cell dysfunction. Recently, plant-derived polyphenols have been the subject of burgeoning interest due to their potent antioxidant and osteogenic activities, but dietary intake reduces targeted therapeutic potential due to their low bioavailability. Thus, there is a technical need for new compositions that can serve as antioxidant and iron chelating therapeutics and/or therapeutics for age-related impaired regeneration in fractures and beyond.

### SUMMARY

Aspects of the present disclosure concern a nanoparticle comprising a plurality of polymers. In certain disclosed aspects, each polymer of the plurality of polymers independently has a structure according to Formula I:

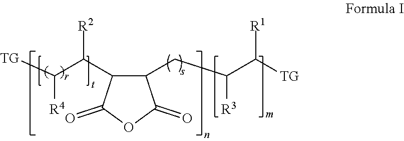

where m, n, and t, independently are integers ≥1; each TG independently is a terminating group; each R¹, R², R³, and R⁴, independently, for each occurrence, is H or comprises an aryl group; each r independently, for each occurrence, is 1 or 0; and each s independently, for each occurrence, is 1 or 0; and provided that, (i) at least one R¹, R², R³, or R⁴, is not, or is other than, phenyl, (ii) one of R¹ or R³, for each occurrence, comprises the aryl group, and (iii) one of R² or R⁴, for each occurrence, comprises the aryl group.

Certain disclosed aspects concern a method of synthesizing a nanoparticle. In certain disclosed aspects, the method comprises preparing a plurality of polymers; preparing a solution of the plurality of polymers in acetone; and dialyzing the solution against water, whereby polymers of the plurality of polymers form into nanoparticles.

Certain disclosed aspects concern a method of using the nanoparticle according to aspects of the disclosure, the method comprising administering a nanoparticle or a pharmaceutically acceptable composition thereof to a subject having, or at risk of having, inflammation and/or iron-dependent cell death.

These aspects and other objects, features, and advantages of the disclosure will become more apparent from the following detailed description, which proceeds with reference to the accompanying figures.

### BRIEF DESCRIPTION OF THE FIGURES

FIG. 1 is a schematic diagram of iron overload in an aged fracture microenvironment according to aspects of the present disclosure.

FIG. 2A is a schematic diagram of cellular mechanisms underpinning a ferroptosis feedback loop according to aspects of the present disclosure.

FIG. 2B is a schematic diagram illustrating how polymer components of a disclosed nanoparticle (NP) can chelate ferric iron and scavenge hydroxyl radicals, thus inhibiting the Fenton reaction.

FIG. 3A is a reaction scheme for synthesizing and protecting 4-vinylcatechol monomers.

FIG. 3B is a reaction scheme for reversible addition-fragmentation chain transfer (RAFT) polymerization of poly(bis(tert-butyldimethylsilyl)-4-vinylcatechol-co-maleic anhydride)-block-poly(bis(tert-butyldimethylsilyl)-4-vinylcatechol) (PtBDMS₂VCMA-b-PtBDMS₂VC) with excess tBDMS₂VC; while FIG. 3B shows a particular polymer backbone configuration, the present disclosure also contemplates aspects where the polymer backbone is as shown for other formulas provided herein.

FIG. 3C is a reaction scheme for deprotection of PtBDMS₂VCMA-b-PtBDMS₂VC to yield poly(4-vinylcatechol-co-maleic anhydride)-block-poly(4-vinylcatechol) (PCatMA-b-PCat) amphiphilic diblocks for micellar NP assembly; while FIG. 3C shows a particular polymer backbone configuration, the present disclosure also contemplates aspects where the polymer backbone is as shown for other formulas provided herein.

FIG. 4 is a plot of preliminary matrix-assisted laser desorption/ionization (MALDI) results showing successful formation of the PCatMA copolymer according to aspects of the present disclosure.

FIG. 5 is a reaction scheme for the preparation of nanoparticles comprising poly(4-vinylcatechol-co-maleic anhydride)-block-poly(4-vinylcatechol) (PCatMA-b-PCat) diblock copolymers according to aspects of the present disclosure; while FIG. 5 shows a particular polymer backbone configuration, the present disclosure also contemplates aspects where the polymer backbone is as shown for other formulas provided herein.

FIG. 6 is another reaction scheme for the preparation of nanoparticles according to aspects of the present disclosure; while FIG. 6 shows a particular polymer backbone configuration, the present disclosure also contemplates aspects where the polymer backbone is as shown for other formulas provided herein.

FIG. 7A is a UV-visible spectrum of PCat NPs (0.0005 w/v % in Tris buffer (pH 7.4) with 0.5 mM ferric citrate) according to aspects of the present disclosure.

FIG. 7B is a plot of a 1,1-diphenyl-2-picrylhydrazyl (DPPH) radical scavenging assay comparing PCat NPs according to aspects of the present disclosure with a vitamin E TROLOX standard.

FIG. 8A is a plot of a metabolic assay performed after treating MC3T3-E1 cells with NP concentrations up to 550 μg/mL, wherein data are presented as means±SDs.

FIG. 8B is a plot of ferrous-specific probe fluorescence in MC3T3-E1 cells incubated with ferrous sulfate heptahydrate concentrations ranging from 0 to 300 UM for 30 minutes before PCat NP treatment, compared to NP-untreated controls, wherein data are presented as means±SDs and statistical significance was determined using two-way ANOVA with Tukey's post-hoc test, *P<0.05, **P<0.005, and ***P<0.0005.

FIG. 8C is a plot of redox probe fluorescence after incubation of MC3T3-E1 cells in H₂O₂, with and without PCat NP treatment, wherein data are presented as means±SDs and statistical significance was determined using two-way ANOVA with Tukey's post-hoc test, *P<0.05, **P<0.005, and ***P<0.0005.

FIG. 8D is a plot of lipid peroxidase probe fluorescence in MC3T3-E1 cells, incubated with and without PCat NPs, wherein data are presented as means±SDs and statistical significance was determined using two-way ANOVA with Tukey's post-hoc test, *P<0.05, **P<0.005, and ***P<0.0005.

FIG. 9A is a plot of a metabolic assay performed after treating RAW 264.7 macrophages with PCat NPs, wherein data are presented as means±SDs.

FIG. 9B is a plot of lactate dehydrogenase (LDH) release from RAW 264.7 macrophages, wherein data are presented as means±SDs.

FIG. 9C is a plot of intracellular ROS concentration with (“+NP”) and without (“−NP”) PCat NP treatment, wherein data are presented as means±SDs.

FIG. 9D is a plot of cytosolic ferrous iron concentration with (“+NP”) and without (“−NP”) PCat NP treatment, wherein data are presented as means±SDs.

FIG. 10 shows two NMR spectra of polymers illustrating incorporation of styrene monomers according to aspects of the present disclosure.

FIG. 11 shows flow cytometry data illustrating cellular uptake of a fluorophore from fluorophore-loaded PCat NPs according to aspects of the present disclosure.

### DETAILED DESCRIPTION

### Explanation of Terms

The following explanations of terms and abbreviations are provided to better describe the present disclosure and to guide those of ordinary skill in the art in the practice of the present disclosure. As used herein, “comprising” means “including” and the singular forms “a” or “an” or “the” include plural references unless the context clearly dictates otherwise. The term “or” refers to a single element of stated alternative elements or a combination of two or more elements unless the context clearly indicates otherwise.

Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting. Other features of the disclosure are apparent from the following detailed description and the claims.

The disclosure of numerical ranges should be understood as referring to each discrete point within the range, inclusive of endpoints, unless otherwise noted. Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise. Functional groups described and/or defined herein can be substituted or unsubstituted unless expressly stated otherwise.

Definitions of common terms in chemistry that are not otherwise defined herein may be found in Richard J. Lewis, Sr. (ed.), Hawley's Condensed Chemical Dictionary, published by John Wiley & Sons, Inc., 2016 (ISBN 978-1-118-13515-0).

In order to facilitate review of the various embodiments of the disclosure, the following explanations of specific terms are provided:

Amphiphilic: Including both a hydrophilic portion and a hydrophobic portion.

Aryl: A monovalent aromatic carbocyclic group of, unless specified otherwise, from 6 to 15 carbon atoms having a single ring (e.g., phenyl) or multiple condensed rings in which at least one ring is aromatic (e.g., naphthalene, anthracene, and the like), provided that the point of attachment is through an atom of an aromatic portion of the aryl group and the aromatic portion at the point of attachment contains only carbons in the aromatic ring. Aryl groups can be monocyclic, bicyclic, tricyclic, or tetracyclic. Aryl groups can be substituted or unsubstituted.

Chelate: A coordination compound in which a central metal ion is attached by coordinate bonds to two or more nonmetal atoms of a single molecule or ligand. The chelate may have a cyclic structure. As a verb, to form a chelate.

Conjugate: Two or more moieties directly or indirectly coupled together. For example, a first moiety may be covalently or noncovalently (e.g., electrostatically) coupled to a second moiety. Indirect attachment is possible, such as by using a “linker” (a molecule or group of atoms positioned between two moieties).

Copolymer: A polymer formed from polymerization of two or more different monomers.

Derivative: A compound that is derived from a similar compound or a compound that can be imagined to arise from another compound, for example, if one atom is replaced with another atom or group of atoms. The latter definition is common in organic chemistry. In biochemistry, the word is used for compounds that at least theoretically can be formed from the precursor compound.

Diblock copolymer has a first region with a first series of one or more monomers and a second region with a different series of one or more monomers. Each series may include a single type of monomer, or may include two or more different monomers, such as two different monomers in a repeating arrangement.

Electron-withdrawing group (EWG): An atom or functional group that decreases electron density at another location in a molecule by attracting electrons toward itself (e.g., through inductive or resonance effects). In some aspects, EWGs can modulate reactivity in organic synthesis and influence the electronic properties of compounds in medicinal chemistry and materials science. For example, EWGs can increase acidity of nearby protons. In some aspects, the EWG accepts electron density from an aromatic ring or olefin group to which it is directly attached, such as by inductive electron withdrawal. Representative and non-limiting examples of electron-withdrawing groups can include certain carbonyl-containing groups (e.g., aldehyde, ketone, ester, carboxylic acid, acyl), a quaternary amine, acyl halide, cyano, sulfonate, nitro, nitroso, pyridinyl, alkyl halide, halogen (e.g., chloro, bromo, fluoro, or iodo), haloaliphatic, ammonium, or amide.

Excipient: A physiologically inert substance that is used as an additive in a pharmaceutical composition.

Ferroptosis: A type of cell death that is iron-dependent.

Initiator: An agent used to start polymerization of a monomer. The initiator typically is consumed in the reaction. In polymerization, the initiator typically forms a free radical to initiate the reaction. Initiators include, by way of example and without limitation, halogens, azo compounds, and organic peroxides. In some aspects, the initiator (or a fragment/functional group of the initiator) can serve as a terminating group in formulas provided herein.

Micellar structure: As used herein, the term “micellar structure” refers to a nanoparticle comprised of amphiphilic polymers wherein, in an aqueous or polar environment, hydrophobic portions of the amphiphilic polymers are oriented towards a center of the nanoparticle and hydrophilic portions of the amphiphilic polymers are oriented towards an outer surface of the nanoparticle. In some representative aspects, the hydrophobic portion is proximate a poly(4-vinylcatechol) (PCat) block of the disclosed diblock copolymers and the hydrophilic portion comprises a poly(4-vinylcatechol-co-maleic anhydride) (PCatMA) block of the disclosed diblock copolymers.

Microenvironment: The immediate small-scale environment of an organism or a part of an organism, especially as a distinct portion of a larger environment, e.g., the cells, molecules and structures surrounding a cell or tissue of interest.

Modulate: To adjust, control, or change a physical process.

Monomer: A molecule or compound, usually containing carbon, that can react and combine to form polymers. For example, polystyrene is formed by polymerization of the monomer styrene. Molecules formed by the combination of monomers can be characterized by the number of monomers. For example, a dimer is a molecule formed from two monomers, a trimer is a molecule formed from three monomers, etc. A molecule with more than 10 monomers is typically referred to using the number of monomeric units, e.g., a 20-mer is a molecule having 20 monomeric units.

Nanoparticle: A nanoscale particle with a size that is measured in nanometers, for example, a nanoscopic particle that has at least one dimension of less than about 100 nm.

Number average molecular weight: The statistical average molecular weight of all the polymer chains in a sample, defined as

$$Mn = \frac{\sum {N}_{i} {M}_{i}}{\sum {N}_{i}} ,$$

where Mᵢ is the molecular weight of a chain, and Nᵢ is the number of chains of that molecular weight. Mₙ can be predicted by polymerization mechanisms and is measured by methods that determine the number of molecules in a sample of a given weight, e.g., gel permeation chromatography, viscometry, colligative methods such as vapor pressure osmometry, end group assays, or proton NMR.

Pharmaceutically acceptable: A substance that can be taken into a subject without significant adverse toxicological effects on the subject.

Polydispersity: A measure of the size distribution or heterogeneity of particle sizes in a population of particles.

Polydispersity index: A measure of the broadness of molecular weight distribution, which is calculated as the ratio of weight average molecular weight to number average molecular weight.

Polymer: A molecule of repeating structural units (e.g., monomers) formed via a chemical reaction, i.e., polymerization.

Reactive oxygen species (ROS): A group of highly reactive molecules containing oxygen, e.g., hydroperoxide, superoxide, hydroxyl radical, and singlet oxygen.

Reversible addition-fragmentation chain transfer (RAFT) polymerization: RAFT polymerization utilizes a chain transfer agent (e.g., a thiocarbonylthio compound) to provide control over molecular weight and polydispersity during free-radical polymerization. RAFT polymerization can be used to generate polymers according to the present disclosure.

Scavenge: To remove or deactivate unwanted chemical species, e.g., reactive oxygen species.

Subject: An animal (human or non-human) subjected to a treatment, observation, or experiment. Includes both human and veterinary subjects, including human and non-human mammals, such as rats, mice, cats, dogs, pigs, horses, cows, and non-human primates.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again, solely by way of example and without limitation, a long-chain hydrocarbon may have a hydroxyl group bonded thereto. Groups which are substituted (e.g., substituted alkyl), may in some embodiments be substituted with a group which is substituted (e.g., substituted aryl). In some embodiments, the number of substituted groups linked together is limited to two (e.g., substituted alkyl is substituted with substituted aryl, wherein the substituent present on the aryl is not further substituted). In some embodiments, a substituted group is not substituted with another substituted group (e.g., substituted alkyl is substituted with unsubstituted aryl).

$$Mw = \frac{\sum {N}_{i} {M}_{i}^{2}}{\sum {N}_{i} {M}_{i}} ,$$

Weight average molecular weight: where Mᵢ is the molecular weight of a chain, and Nᵢ is the number of chains of that molecular weight. Weight average molecular weight is determined by methods that are sensitive to molecular size, such as light-scattering techniques, small angle neutron scattering, X-ray scattering, and sedimentation velocity.

### Abbreviations

AIBN: 2,2′-azo-bisisobutryonitrile
Alloc: allyloxycarbonyl
ALP: alkaline phosphatase
ARO: anhydride ring-opening
BMD: bone mineral density
BMDMs: bone-marrow-derived macrophages
CT: computed tomography
CTA: chain-transfer agent
DCFH-DA: diacetyldichlorofluorescein
DCT: 4-cyano-4-[(dodecylsulfanylthiocarbonyl) sulfanyl]pentanoic acid
DOE: design of experiments
DLS: dynamic light scattering
DP: degree of polymerization
DPPH: 1,1-diphenyl-2-picrylhydrazyl
EDC: 1-ethyl-3-(3-dimethylaminopropyl) carbodiimide
ELISA: enzyme-linked immunosorbent assays
EWG: electron-withdrawing group
FAC: ferric ammonium citrate
FACS: fluorescence-activated cell sorting
GPC: gel permeation chromatography
GPX4: glutathione peroxidase 4
GSK-3: glycogen synthase kinase 3
ICA: icariin
LDH: lactate dehydrogenase
M1_{FAC}: excess iron treatment with ferric ammonium citrate for 48 h
M1_{LPS}: treatment with LPS and interferon-γ for 48 h
M2: treatment with interleukin-4 for 48 h
MA: maleic anhydride
MALDI: matrix-assisted laser desorption/ionization
Mₙ: number average molecular weight
MDA: malondialdehyde
MRI: magnetic resonance imaging
MWCO: molecular weight cutoff
NMR: nuclear magnetic resonance
NP: nanoparticle
NTA: nanoparticle tracking analysis
OPF: osteoporotic fracture
Ovx: ovariectomy treatment group
PBS: phosphate buffer solution
PDI: polydispersity
PtBDMS₂VC: poly(bis(tert-butyldimethylsilyl)-4-vinylcatechol)
PtBDMS₂VCMA: poly(bis(tert-butyldimethylsilyl)-4-vinylcatechol-co-maleic anhydride)
PtBDMS₂VCMA-b-PtBDMSzVC: poly(bis(tert-butyldimethylsilyl)-4-vinylcatechol-co-maleic anhydride)-block-poly(bis(tert-butyldimethylsilyl)-4-vinylcatechol)
qPCR: quantitative real-time polymerase chain reaction
RAFT: reversible addition-fragmentation chain transfer
RCF: relative centrifugal force
ROS: reactive oxygen species
SD: standard deviation
tBDMS: tert-butyldimethylsilyl
TBP: bone-targeting peptide
TEAC: TROLOX-equivalent antioxidant capacity
THN: 1,2,3,4-tetrahydronapthalene
TG: terminating group
VC: vinylcatechol

### INTRODUCTION

As introduced above, ferroptosis, a form of iron-dependent inflammatory cell death, occurs when antioxidant pathways are depleted by the iron-catalyzed generation of ROS and phospholipid peroxidation. Ferroptosis has been implicated in a myriad of age-related inflammatory orthopedic, cardiovascular, and neurodegenerative diseases. As cells age, dysregulated iron metabolism results in excess iron accumulation, oxidative damage, and a chronic inflammatory state termed “inflammaging.” There is a pressing need to develop efficacious therapeutics that address the mechanisms underpinning age-related chronic inflammatory diseases.

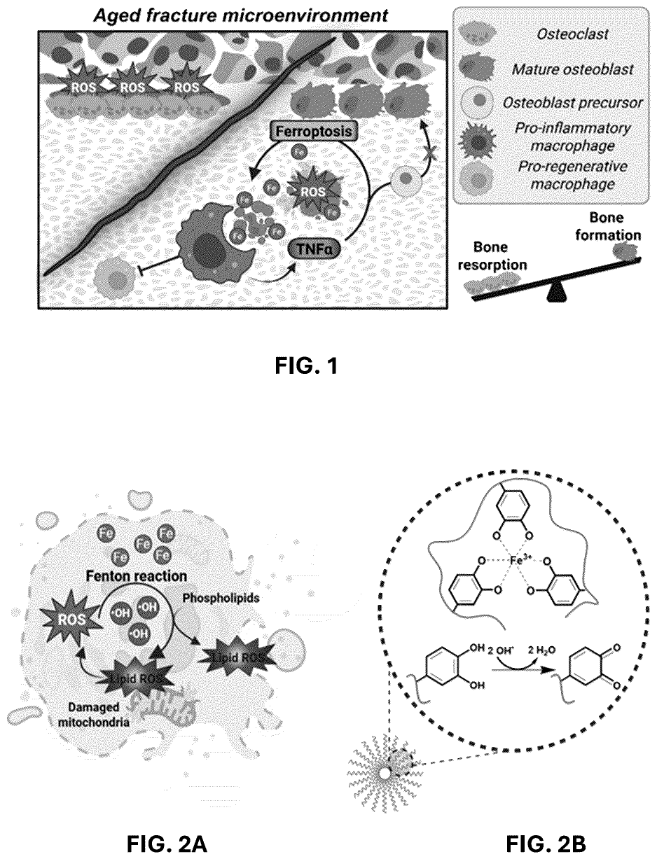

Antioxidant therapeutics capable of scavenging ROS offer a promising avenue for reversing this proinflammatory milieu. For example, iron chelation and antioxidant treatments show promise in treating ferroptosis in age-related fractures. Aged fractures exhibit a chronic inflammatory feedback loop between pro-inflammatory macrophages and bone cells underpinned by iron. Consequently, fracture regeneration is impaired through bone resorption outweighing formation due to sustained pro-inflammatory macrophage polarization, increased osteoclast activity, and ferroptosis of osteoblasts (FIG. 1). As shown in FIG. 1, iron in the aged fracture microenvironment leads to persistent pro-inflammatory macrophage polarization, tumor necrosis factor-alpha (TNFα) secretion, inhibition of osteoblast maturation, and osteoblast ferroptosis. Pro-inflammatory factors (ROS, iron, and TNFα) result in osteoclast activity outstripping osteoblasts, impairing regeneration. Ferroptosis occurs when intracellular antioxidant pathways are depleted due to excess cytosolic iron, generating highly reactive hydroxyl radicals through Fenton and Fenton-like reactions (FIG. 2A).

Combined iron chelation and antioxidant treatments have shown promise in the inhibition of ferroptosis in osteoporotic animal models. However, delivery in vivo remains a significant challenge due to poor plasma half-lives, reduced bioavailability, and off-target side effects. Furthermore, age-associated iron dysregulation is often due to hindered iron mobility, resulting in simultaneous tissue-level iron overload paired with systemic functional anemia. Recently, plant-derived polyphenols have been the subject of burgeoning interest due to their potent antioxidant and osteogenic activities, but dietary intake reduces targeted therapeutic potential due to their low bioavailability.

Disclosed herein are aspects of amphiphilic poly((substituted styrene)-co-maleic anhydride)-block-poly(substituted styrene) diblock copolymers that are synthesized using reversible addition-fragmentation chain transfer (RAFT) polymerization. In particular aspects, polycatecholic (PCat) NPs comprising catechol-containing polyphenols are described, which spontaneously form supramolecular metal-phenolic networks with bivalent and trivalent metal ions. At physiological pH, catechol ligands can undergo tetradentate and hexadentate coordination with ferric iron, removing the catalytic site of the Fenton reaction and preventing reduction to ferrous iron, and scavenging ROS. Furthermore, PCat NPs according to the present disclosure offer an innovative advantage over traditional NP drug-delivery systems due to the potential to leverage their innate therapeutic activity as both a polymeric prodrug and drug delivery vehicle for potential synergistic effects in age-related and/or chronic inflammatory conditions underpinned by ferroptosis.

### Polymers and Nanoparticles

Aspects of the present disclosure concern a nanoparticle that comprises a plurality of polymers. In some aspects, each polymer of the plurality of polymers independently has a structure according to Formula I:

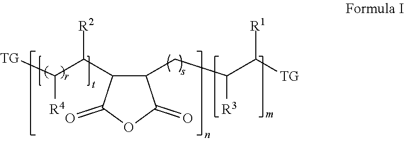

where m, n, and t, independently are integers ≥1; each TG independently is a terminating group; each R¹, R², R³, and R⁴, independently, for each occurrence, is H or comprises an aryl group; each r independently, for each occurrence, is 1 or 0; and each s independently, for each occurrence, is 1 or 0; and provided that, (i) at least one R¹, R², R³, or R⁴, is not, or is other than, phenyl, (ii) one of R¹ or R³, for each occurrence, comprises the aryl group, and (iii) one of R² or R⁴, for each occurrence, comprises the aryl group.

In some aspects, R¹, R², R³, and R⁴, are different chemical moieties.

In some aspects, every “m” bock may have the same R¹ and/or R³ groups. In other aspects, one or more “m” blocks may have different R¹ and/or R³ groups from other “m” blocks.

In some aspects, every “n” bock may have the same R² and/or R⁴ groups. In other aspects, one or more “n” blocks may have different and/or R⁴ groups from other “n” blocks

In some aspects, at least one occurrence of R¹ (or R³) and R² (or R⁴), comprises a phenyl group, provided that at least one occurrence of the remaining R¹, R², R³, or R⁴ groups in the plurality of polymers is not, or is other than, phenyl, and is typically H.

In some aspects, each R¹ and R³, independently, are the same for each occurrence and at least one of these two groups is a phenol group, a dihydroxybenzene group (e.g., a catechol group, a resorcinol group, or a hydroquinone group), a trihydroxybenzene group, a tetrahydroxybenzene group, or a pentahydroxybenzene group and the other is H. In some aspects, each R² or R⁴, independently, are the same for each occurrence and at least one of these two groups is a phenol group, a dihydroxybenzene group (e.g., a catechol group, a resorcinol group, or a hydroquinone group), a trihydroxybenzene group, a tetrahydroxybenzene group, or a pentahydroxybenzene group and the other is H.

In some aspects, each R¹ and R³, independently, are the same for each occurrence and at least one of these groups comprises a chelating group and the other is H. In some aspects, each R² and R⁴, independently, are the same for each occurrence and at least one of these groups comprises a chelating group and the other is H. For example, the hydroxide functional groups on a catechol group can bind metals, such as iron. This can reduce ion concentration in an environment of the polymer (e.g., in or around a bone cell).

In some aspects, one of R¹ or R³ independently, for each occurrence, comprises an aryl ring which comprises an electron-withdrawing group on the aryl ring and the other of R¹ or R³ is H; and one of R² or R⁴ independently, for each occurrence, comprises an aryl ring which comprises an electron-withdrawing group on the aryl ring and the other of R² or R⁴ is H. In some aspects, the electron-withdrawing group is a halogen, a nitro group, a cyano group, or a carbonyl-containing group. In certain aspects, the electron-withdrawing group is a halogen or a nitro group. In certain aspects, the electron-withdrawing group can modulate the pKa of a nucleophilic chelating group (e.g., a hydroxy group) on the aryl ring and result in stronger binding to a target metal (e.g., iron).

In some aspects, m is an integer ≥10, ≥25, ≥50, ≥75, ≥100, such as from 10 to 500, from 25 to 500, from 50 to 500, from 75 to 500, from 75 to 400, or from 75 to 300.

In some aspects, n is an integer ≥10, ≥25, ≥40, such as from 10 to 500, from 10 to 300, from 25 to 300, from 40 to 300, or from 40 to 250.

In some aspects, t is an integer selected from 1 to 10, such as 1, 2, 3, 4, 5, 6, 7, 8, 9, or 10.

In particular aspects, each r is the same and is 1. In yet other aspects, each r is the same and is 0.

In particular aspects, each s is the same and is 1. In yet other aspects, each s is the same and is 0.

In some aspects, the polymers have a number average molecular weight of from 10 kDa to 150 kDa, from 20 kDa to 80 kDa, from 20 kDa to 70 kDa, from 20 kDa to 60 kDa, from 20 kDa to 50 kDa, or from 20 kDa to 40 kDa, as determined by gel permeation chromatography or nuclear magnetic resonance spectroscopy calculation.

In some aspects, the terminating groups are provided by an initiator, a chain transfer agent, or a combination thereof. In certain aspects, each TG is independently selected from a cyano-containing functional group or a —SC(S)S-containing functional group. In certain aspects, one TG is

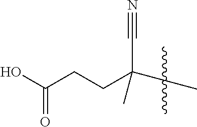 [SMILES: *C(C)(C#N)CCC(=O)O]

and the other TG is

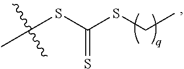

wherein q is an integer ranging from 0 to 20. An example of a suitable chain transfer agent is 4-cyano-4-[(dodecylsulfanylthiocarbonyl) sulfanyl]pentanoic acid. An example of a suitable initiator is 2,2′-azo-bisisobutryonitrile (AIBN).

In some aspects, each polymer of the plurality of polymers according to Formula I may independently have a structure according to Formula l′, Formula I″, or Formula I″ wherein Ar represents an aryl group as described herein.

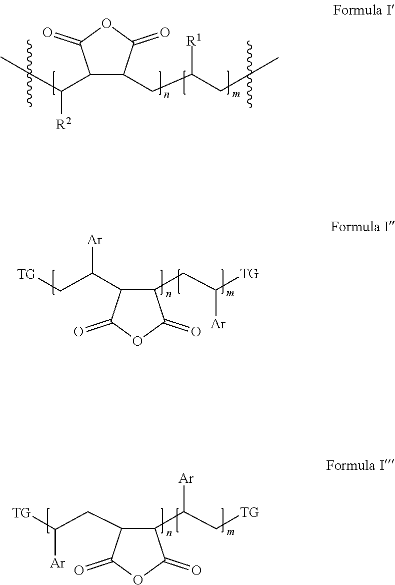

In certain aspects, each polymer of the plurality of polymers independently has a structure according to Formula IA, Formula IA′, or Formula IA″:

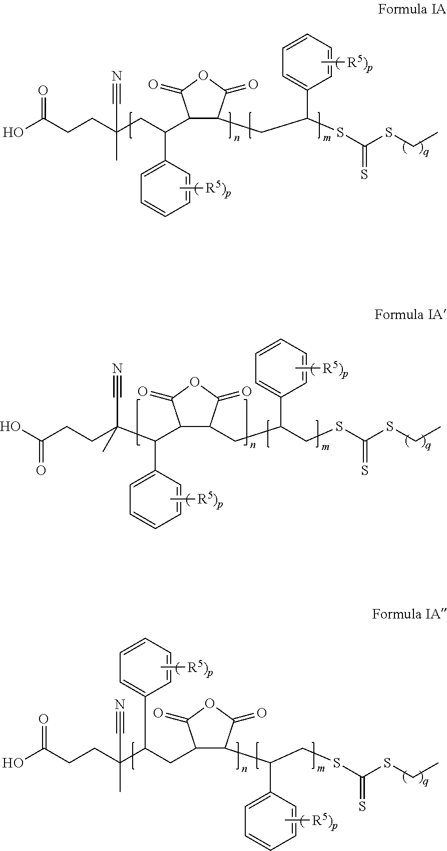

where m and n independently are integers ≥1; wherein each R⁵ independently, for each occurrence, is selected from —OH, a halogen, or a nitro group; each p, for each occurrence, is an integer selected from 0 to 5; and q is an integer selected from 0 to 20.

In some aspects, one or more of the polymers comprise alternating units in a repeating AB pattern. In some aspects, the “A” units comprise an R¹-containing monomer and the “B” units comprise a monomer comprising a maleic anhydride and an R² group. In some aspects, the “A” units are derived from polymerization of (R¹ and/or R³)-containing monomers, and the “B” units are derived from polymerization of monomers comprising a maleic anhydride group and an (R² and/or R⁴) group. In some aspects, the alternating copolymeric architecture of the AB polymers forms spontaneously due to the complementary electron densities of the “A” unit and “B” unit monomers.

In certain aspects, the alternating units in the repeating AB pattern are interspersed with C units derived from polymerization of styrene monomers or other (R¹ and/or R³)-containing groups, which are different from the A units. For example, one or more of the polymers may comprise alternating units in a repeating ABC pattern, or a repeating AB pattern where C is occasionally introduced into the polymer structure (e.g., ABABCAB). This can, in some aspects, modulate chelation by introducing varying types of chelating functional groups, by tuning the concentration of chelating functional groups in the polymer, by tuning the conformational flexibility of the polymer, or a combination thereof.

In some aspects, if the oxidative stability of PCat NPs is low (as measured by the oxidation of polyphenolic protons via spectroscopic analysis), bioinert ROS- and pH-labile boronate ester groups can be conjugated onto hydroxyl groups of the polymer backbone (e.g., hydroxyl groups bound to the R¹ or R² aryl rings) to maintain antioxidant capacity before treatment. In some aspects, if adequate iron chelation capacity at cytocompatible concentrations proves difficult, the polymers may be structurally modified to increase conformational flexibility and/or coordination complex stability.

In some aspects, the plurality of polymers comprises poly(4-vinylcatechol-co-maleic anhydride)-block poly(4-vinylcatechol) (PCatMA-b-PCat) diblock copolymers. In some such aspects, each polymer comprises a poly(4-vinylcatechol-co-maleic anhydride) (PCatMA) block and a poly(4-vinylcatechol) (PCat) block.

In some aspects, a PCatMA block comprises alternating units in a repeating AB pattern derived from polymerizing 4-vinylcatechol and maleic anhydride, wherein the 4-vinylcatechol can make up the A units and a combination of maleic anhydride and 4-vinylcatechol can make up the B units.

In certain aspects, each polymer of the plurality of polymers independently has a structure according to Formula IIB, Formula IIB′, or Formula IIB″:

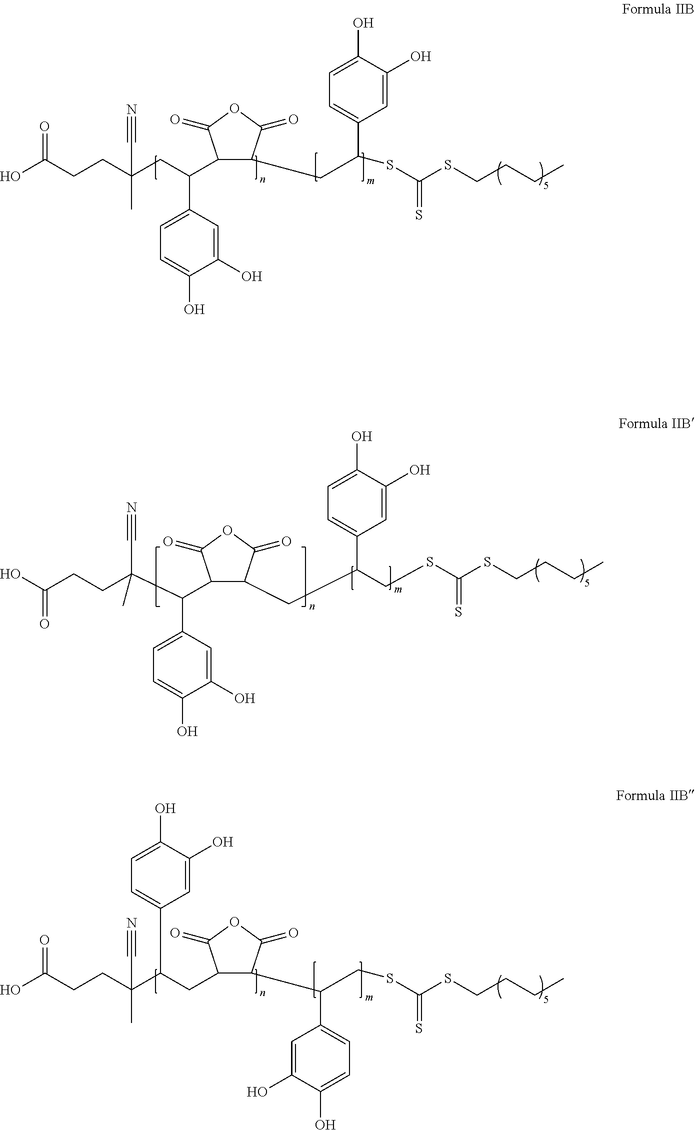

where m and n independently are integers ≥1.

In certain aspects, the PCatMA blocks have a number average molecular weight of from 10 kDa to 75 kDa, from 15 kDa to 50 kDa, from 20 kDa to 40 kDa, or from 20 kDa to 35 kDa, as determined by gel permeation chromatography or nuclear magnetic resonance (NMR) spectroscopy calculation.

In certain aspects, the PCat blocks have a number average molecular weight of from 10 kDa to 75 kDa, from 10 kDa to 45 kDa, from 15 kDa to 40 kDa, from 15 kDa to 30 kDa, or from 20 kDa to 30 kDa, as determined by gel permeation chromatography or NMR spectroscopy calculation.

In some aspects, the polymer chains have a size distribution with a polydispersity index of from 1.0 to 1.6, as determined by the ratio of weight average molecular weight to number average molecular weight. In certain aspects, the polydispersity index ranges from 1.05 to 1.50. In certain aspects, the polydispersity index ranges from 1.05 to 1.40.

In some aspects, the polymers are amphiphilic with a hydrophobic portion and a hydrophilic portion. In some aspects, the maleic anhydride-containing portion of the polymer of Formula I (the portion of Formula I in brackets “[ ]” that is repeated n times) is hydrophilic, while R¹, R², R³, R⁴ or a combination thereof, contain hydrophobic functional groups (e.g., aryl groups). In particular aspects, the block containing R¹ and/or R³ (the portion of Formula I in brackets “[ ]” that is repeated m times) is more hydrophobic than the maleic anhydride-containing portion of the polymer. Accordingly, in certain aspects, the polymers can self-assemble into a nanoparticle having a micellar structure, such that the hydrophobic portion is oriented toward a center of the nanoparticle and the hydrophilic portion is oriented toward an outer surface of the nanoparticle. In some aspects, and as described in more detail below, the amphiphilic nature of the polymers also enables encapsulation of another molecule (e.g., a therapeutic agent or a detectable marker) within the hydrophobic core. In some aspects, the hydrophilic aspects of the maleic anhydride-containing backbone can serve as a handle for post-synthetic functionalization.

In certain aspects, where the plurality of polymers comprises a PCatMA block and a PCat block, the hydrophobic portion is proximate the PCat block and the hydrophilic portion comprises the PCatMA block. Accordingly, in certain aspects, the PCat blocks of the polymers are oriented toward a center of the nanoparticle and the PCatMA blocks of the polymers are oriented toward an outer surface of the nanoparticle.

In some aspects, a plurality of the nanoparticles has an average diameter of from 40 nm to 180 nm, as determined by DLS. In certain aspects, the diameter ranges from 80 nm to 180 nm. In certain aspects, the diameter ranges from 80 nm to 170 nm.

In some aspects, a plurality of the nanoparticles has a polydispersity of 0.05 to 0.50, as determined by DLS. In certain aspects, the polydispersity ranges from 0.05 to 0.40. In certain aspects, the polydispersity ranges from 0.05 to 0.30.

In some aspects, the nanoparticle comprises a core defined by the plurality of polymers. In certain aspects, a substance is encapsulated within the core. In certain aspects, the substance is a therapeutic agent, a detectable label, or a combination thereof, which is encapsulated in the core. Non-limiting examples of drugs that can be encapsulated within the core of the nanoparticle include a glycogen synthase kinase 3 (GSK-3) inhibitor (such as AZD2858, also referred to as AR28), an iron chelator (e.g., 8-hydroxyquinoline, tropolone, pyridoxal isonicotinoyl hydrazone), ferrostatin-1, and an antioxidant/iron-binding molecule (e.g., quercetin, luteolin, tannic acid, myrcetin, epigallocatechin-3-gallate). Non-limiting examples of detectable labels that can be encapsulated within the core of the nanoparticle include a colored or fluorescent imaging agent, a radioisotope, or a contrast agent for medical imaging (e.g., a magnetic resonance imaging (MRI) contrast agent or a computed tomography (CT) contrast agent).

In some aspects, the nanoparticle is coupled to a moiety, such as a protein, another compound (such as a therapeutic agent, a detectable label, or a targeting ligand), or a combination thereof. In particular aspects, the nanoparticle is chemically coupled to the moiety. In certain aspects, the moiety is a targeting ligand, which comprises a binding peptide configured to bind the nanoparticle to a target. In particular aspects, the targeting ligand comprises a bone-binding peptide (TBP), which can be conjugated to an outer portion of the nanoparticle through the maleic anhydride functionality. In some aspects, as depicted in Scheme 1 below, allyloxycarbonyl (Alloc)-protected TBP is coupled to a polymer at the maleic anhydride functionality, then deprotected to yield a TBP-functionalized polymer. While Scheme 1 shows a particular polymer backbone configuration, the present disclosure also contemplates aspects where the polymer backbone is as shown for other formulas provided herein.

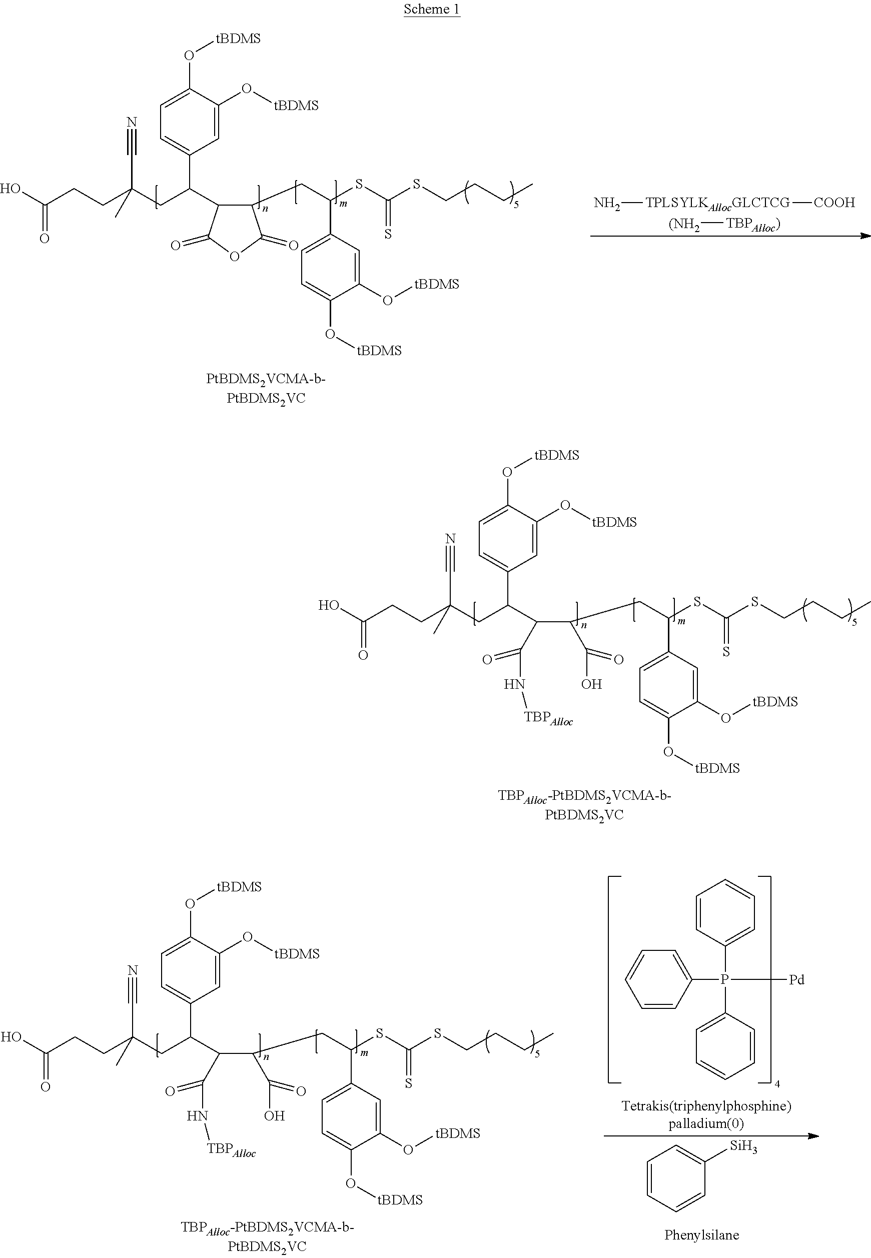

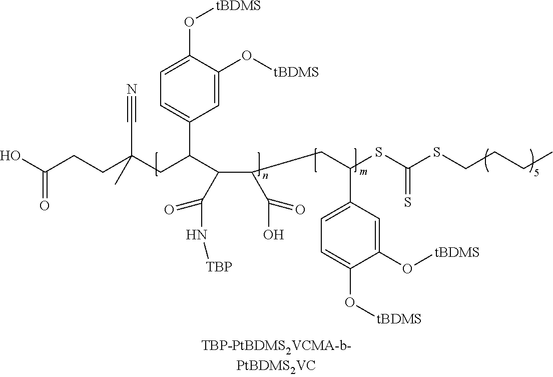

In some additional aspects, the moiety is a targeting ligand that comprises a transferrin binding protein or a transferrin-mimetic peptide. In this manner, the nanoparticles are able to bind multiple transferrin molecules in vivo (and/or bind to iron-hungry cells in a manner that mimics transferrin, in the case of a NP conjugated to a transferrin-mimetic peptide), thereby increasing the likelihood that the nanoparticle will be taken up by iron-hungry cells.

In yet other aspects, the moiety is a targeting ligand that comprises hepcidin or a hepcidin-mimetic peptide. In this manner, the nanoparticles are able to bind multiple hepcidin molecules in vivo in vivo (and/or bind to iron transport channels in a manner that mimics hepcidin, in the case of a NP conjugated to a hepcidin-mimetic peptide).

In some aspects, the moiety is a therapeutic agent selected from an analgesic, an anesthetic, an antifungal, an antibiotic, an anti-inflammatory, an anthelmintic, an antidote, an antiemetic, an antihistamine, an antihypertensive, an antimalarial, an antimicrobial, an antipsychotic, an antipyretic, an antiseptic, an antiarthritic, an antituberculotic, an antitussive, an antiviral, a cardioactive drug, a cathartic, a chemotherapeutic agent, a corticoid such as a steroid, an antidepressant, a depressant, a diagnostic aid, a diuretic, an enzyme, an expectorant, a hormone, a hypnotic, a mineral, a nutritional supplement, a parasympathomimetic, a potassium supplement, a radiation sensitizer, a radioisotope, a sedative, a sulfonamide, a stimulant, a sympathomimetic, a tranquilizer, a urinary anti-infective, a vasoconstrictor, a vasodilator, a vitamin, a xanthine derivative, or any combination thereof. In some aspects, the therapeutic agent comprises another small organic molecule, a naturally isolated entity or its analog, an organometallic agent, a chelated metal or metal salt, a peptide-based drug, or a peptidic or non-peptidic receptor targeting or binding agent. In some aspects, the therapeutic agent comprises a vitamin or other nutritional supplement, a glycoprotein such as collagen, fibronectin, a peptide or protein, a carbohydrate (simple and/or complex), a proteoglycan, an antigen, an oligonucleotide (sense and/or antisense DNA and/or RNA), an antibody (for example, to infectious agents, tumors, drugs, or hormones), a gene therapy reagent, or any combination thereof.

In certain aspects, the nanoparticle is coupled to an iron-chelating drug. One example of a suitable iron-chelating drug is deferoxamine. Molecules of deferoxamine contain a primary amine moiety, which can react with the maleic anhydride moiety of the polymer to thereby conjugate the deferoxamine to the nanoparticle.

Moieties discussed above can be coupled to the nanoparticle in any suitable manner recognized by those in the art with the benefit of the present disclosure. For example, conjugation chemistry approaches can be used, including but not limited to amine-carboxyl linkers, amine-sulfhydryl linkers, amine-carbohydrate linkers, amine-hydroxyl linkers, amine-amine linkers, carboxyl-sulfhydryl linkers, carboxyl-carbohydrate linkers, carboxyl-hydroxyl linkers, carboxyl-carboxyl linkers, sulfhydryl-carbohydrate linkers, sulfhydryl-hydroxyl linkers, sulfhydryl-sulfhydryl linkers, carbohydrate-hydroxyl linkers, carbohydrate-carbohydrate linkers, and hydroxyl-hydroxyl linkers. In some aspects, the moiety is a therapeutic agent that is linked to the polymer by a protease-sensitive linker or other biodegradable linkage. In certain aspects, “click” chemistry (e.g., azide-alkyne cycloaddition) is used to attach a moiety to the polymers of the nanoparticle. In some aspects, a moiety is first attached to a monomer and the resulting compound is then used in the polymerization synthesis of a polymer that is used to form the nanoparticle.

In certain aspects, the moiety is coupled to the nanoparticle by hydrolysis of a maleic anhydride moiety in the polymer, followed by carbodiimide coupling of an amine to the exposed carboxylic acids of the hydrolyzed maleic anhydride.

In certain aspects, the moiety is coupled to the nanoparticle by anhydride ring-opening (ARO) nucleophilic addition-elimination.

In certain aspects, the moiety is coupled to the nanoparticle using a boronic acid derivative or by attaching the compound to the boronic acid derivative (see Scheme 2). While Scheme 2 shows a particular polymer backbone configuration, the present disclosure also contemplates aspects where the polymer backbone is as shown for other formulas provided herein.

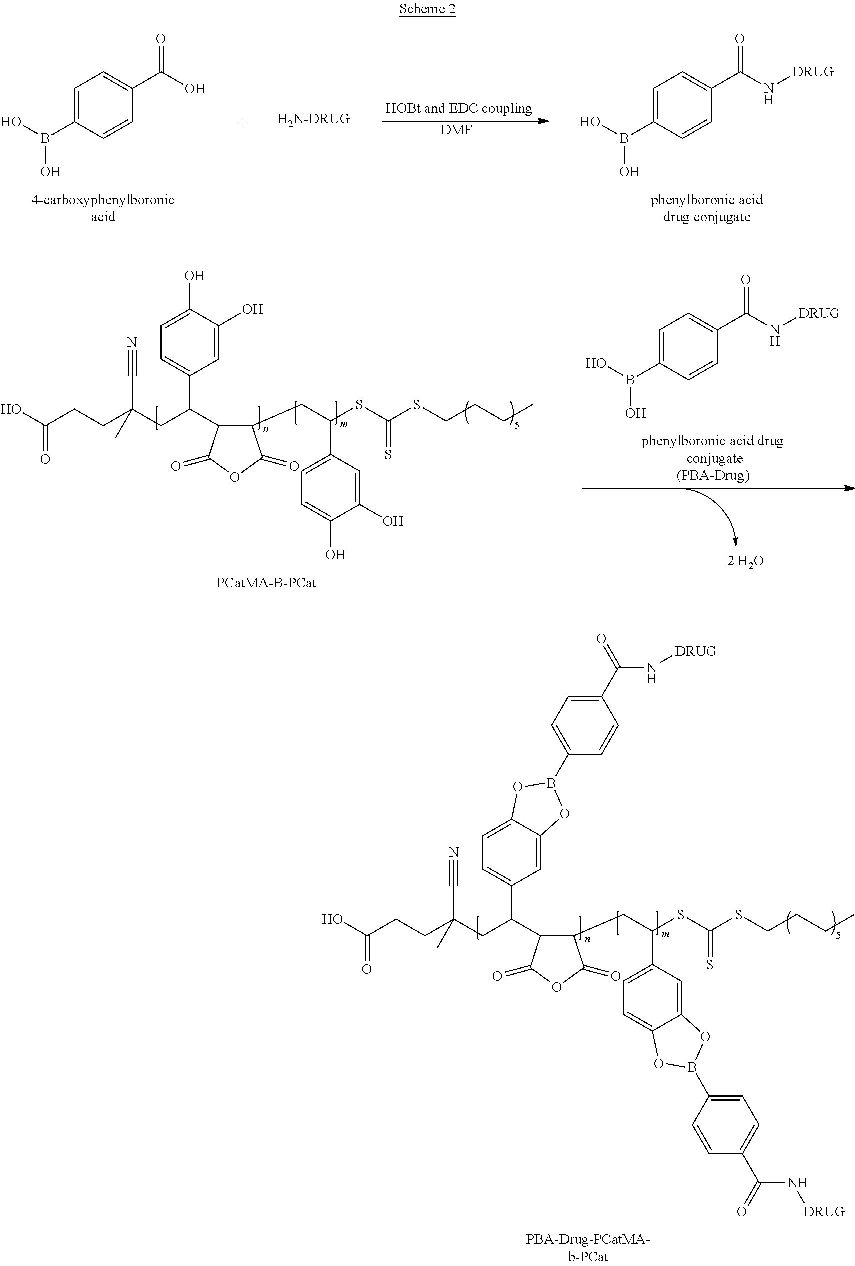

In particular aspects, deferoxamine can be conjugated to 4-carboxylic phenyl boronic acid by 1-ethyl-3-(3-dimethylaminopropyl) carbodiimide (EDC) coupling to form catecholate boronic esters with the catechol groups in the polymer of Formulas IIB, IIB′, and IIB″. This may serve as a mechanism to couple one or more moieties to the NP, while protecting the polymer from premature oxidative degradation in vivo. Furthermore, this may enable the NPs to act as a source for stimuli-responsive drug delivery, as the boronic esters are cleaved in acidic and ROS rich intracellular environments.

### Method of Synthesizing Nanoparticles

Aspects of the present disclosure concern a method of synthesizing a nanoparticle according to aspects of the present disclosure.

In some aspects, the method comprises preparing a plurality of polymers.

In certain aspects, each polymer of the plurality of polymers has a structure according to Formula I:

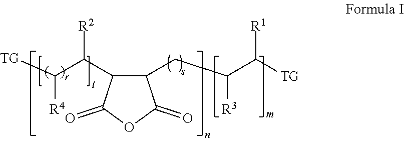

where m, n, and t, independently are integers ≥1; each TG independently is a terminating group; each R¹, R², R³, and R⁴, independently, for each occurrence, is H or comprises an aryl group; each r independently, for each occurrence, is 1 or 0; and each s independently, for each occurrence, is 1 or 0; and provided that, (i) at least one R¹, R², R³, or R⁴, is not, or is other than, phenyl, (ii) one of R¹ or R³, for each occurrence, comprises the aryl group, and (iii) one of R² or R⁴, for each occurrence, comprises the aryl group. In certain aspects, each TG is independently selected from a cyano-containing functional group or a —SC(S)S-containing functional group. In particular aspects, one TG is

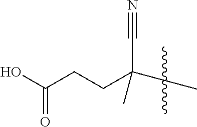 [SMILES: *C(C)(C#N)CCC(=O)O]

and the other TG is

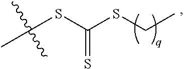

wherein q is an integer ranging from 0 to 20.

In certain aspects, each polymer of the plurality of polymers has a structure according to any one of Formula l′, I″, I′″ IA, IA′, IA″, IIB, IIB′, or IIB″.

In certain aspects, preparing the plurality of polymers comprises combining maleic anhydride and an R¹- and/or R²-containing compound (e.g., a vinyl aryl monomer having a structure according to a formula CH₂═CH-Aryl) with a chain transfer agent, an initiator, and a solvent to form a monomer solution.

In certain aspects, preparing the plurality of polymers comprises combining maleic anhydride and O-bis(tert-butyldimethylsilyl)-4-vinylcatechol with a chain transfer agent, an initiator, and a solvent to form a monomer solution.

In certain aspects, a molar ratio of maleic anhydride to O-bis(tert-butyldimethylsilyl)-4-vinylcatechol ranges from 1:1 to 1:5, from 1:2 to 1:5, from 1:2 to 1:4, or from 1:2.5 to 1:3.5.

In certain aspects, the chain transfer agent is 4-cyano-4-[(dodecylsulfanylthiocarbonyl) sulfanyl]pentanoic acid.

In certain aspects, the initiator is 2,2′-azo-bisisobutryonitrile.

In certain aspects, the solvent comprises dioxane, toluene, or a combination thereof.

In certain aspects, the method further comprises heating the monomer solution at a temperature effective to induce polymerization and produce the plurality of polymers. In certain aspects, the temperature is also effective to remove protecting groups.

In certain aspects, the effective temperature is from 50° C. to 70° C., from 55° C. to 65° C., or 60° C.

In some aspects, the method comprises preparing a solution of the plurality of polymers in acetone.

In some aspects, the method further comprises dialyzing the solution against water. In certain aspects, this dialyzing causes the plurality of polymers to form into nanoparticles.

In certain aspects, the nanoparticles have a micellar structure. In certain aspects, the polymers are amphiphilic with a hydrophobic portion and a hydrophilic portion. In this manner, the polymers can self-assemble into nanoparticles such that the hydrophobic portion is oriented toward a center of the nanoparticle and the hydrophilic portion is oriented toward an outer surface of the nanoparticle.

In certain aspects, where the plurality of polymers comprise a PCatMA block and a PCat block, the hydrophobic portion is proximate the PCat block and the hydrophilic portion comprises the PCatMA block.

In some aspects, preparing the solution of the polymers in acetone further comprises adding a therapeutic agent and/or a detectable label to the solution. In certain aspects, dialyzing the solution against water provides nanoparticles encapsulating the therapeutic agent and/or the detectable label.

### Pharmaceutical Formulations and Method of Use

The nanoparticles described herein can be used to prepare therapeutic pharmaceutical compositions. In some aspects, the nanoparticles described herein, and compositions thereof, can be used to treat a subject, particularly to treat inflammation, iron-dependent cell death (e.g., ferroptosis), or a combination thereof. In additional aspects, the nanoparticles, and compositions thereof, can be used as antioxidants. In some aspects, the polymers, additionally or alternatively, act on ROS, such as to scavenge an ROS. Without being limited to a single theory, it currently is believed that, by chelating iron and (additionally or alternatively) scavenging ROS, the nanoparticle of the present disclosure may mitigate inflammation, iron-dependent cell death (e.g., ferroptosis), or a combination thereof. In this manner, the nanoparticle may increase a rate of healing. For example, when administered to a bone cell (e.g., an osteogenic cell, an osteogenic progenitor cell, an osteoclast, an osteoblast, or an osteocyte), the nanoparticle may increase a rate of bone fracture healing, prevent or mitigate the effects of osteoarthritis, or a combination thereof. In some aspects, the nanoparticle additionally or alternatively prevents or mitigates the effects of age-related chronic inflammation.

The nanoparticles described herein can be formulated as a pharmaceutical composition and administered to a mammalian host, such as a human or veterinary patient, in a variety of forms. The forms can be specifically adapted to a chosen route of administration, e.g., oral or parenteral administration, by intravenous, intramuscular, topical, or subcutaneous routes.

The nanoparticles described herein may be systemically administered in combination with a pharmaceutically acceptable vehicle, such as an inert diluent or an assimilable edible carrier. For oral administration, nanoparticles can be enclosed in hard or soft shell gelatin capsules, compressed into tablets, or incorporated directly into the food of a patient's diet. Nanoparticles may also be combined with one or more excipients and used in the form of ingestible tablets, buccal tablets, troches, capsules, elixirs, suspensions, syrups, wafers, and the like. Such compositions and preparations typically contain at least 0.1% of the nanoparticles. The percentage of the compositions and preparations can vary and may conveniently be from about 2% to about 60% of the weight of a given unit dosage form. The amount of the nanoparticle in such therapeutically useful compositions is such that an effective dosage level can be obtained.

The tablets, troches, pills, capsules, and the like may also contain one or more of the following: binders such as gum tragacanth, acacia, corn starch or gelatin; excipients such as dicalcium phosphate; a disintegrating agent such as corn starch, potato starch, alginic acid and the like; and a lubricant such as magnesium stearate. A sweetening agent such as sucrose, fructose, lactose, or aspartame; or a flavoring agent such as peppermint, oil of wintergreen, or cherry flavoring, may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the nanoparticle, sucrose or fructose as a sweetening agent, methyl and propyl parabens as preservatives, a dye and flavoring such as cherry or orange flavor. Any material used in preparing any unit dosage form should be pharmaceutically acceptable and substantially non-toxic in the amounts employed. In addition, the nanoparticle may be incorporated into sustained-release preparations and devices.

The nanoparticle may be administered intravenously or intraperitoneally by infusion or injection. Solutions of the nanoparticle can be prepared in water, optionally mixed with a nontoxic surfactant. Dispersions can be prepared in glycerol, liquid polyethylene glycols, triacetin, or mixtures thereof, or in a pharmaceutically acceptable oil. Under ordinary conditions of storage and use, preparations may contain a preservative to prevent the growth of microorganisms.

Pharmaceutical dosage forms suitable for injection or infusion can include sterile aqueous solutions, dispersions, or sterile powders comprising the nanoparticle adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions, or by the use of surfactants. The prevention of the action of microorganisms can be brought about by various antibacterial and antifungal agents, for example, parabens, chlorobutanol, phenol, sorbic acid, thiomersal, and the like. In many cases, it will be preferable to include isotonic agents, for example, sugars, buffers, or sodium chloride. Prolonged absorption of the injectable compositions can be brought about by agents delaying absorption, for example, aluminum monostearate and/or gelatin.

Sterile injectable solutions can be prepared by incorporating the nanoparticle in the required amount in the appropriate solvent with various of the other ingredients enumerated above, as required, followed by filter sterilization. In the case of sterile powders for the preparation of sterile injectable solutions, methods of preparation can include vacuum drying and freeze-drying techniques, which yield a powder of the nanoparticle plus any additional desired ingredient present in the previously sterile-filtered solutions.

For topical administration, it will generally be desirable to administer the nanoparticle to the skin as a composition or formulation, for example, in combination with a dermatologically acceptable carrier, which may be a solid or a liquid.

Useful solid carriers include finely divided solids such as talc, clay, microcrystalline cellulose, silica, alumina, and the like. Useful liquid carriers include water, DMSO, alcohols, glycols, or water-alcohol/glycol blends, in which a nanoparticle can be suspended or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using a pump-type or aerosol sprayer.

Thickeners such as synthetic polymers, fatty acids, fatty acid salts and esters, fatty alcohols, modified celluloses, or modified mineral materials can also be employed with liquid carriers to form spreadable pastes, gels, ointments, soaps, and the like, for application directly to the skin of the user.

Examples of dermatological compositions for delivering active agents to the skin are recognizable by those in the art, particularly with the benefit of the present disclosure. Such dermatological compositions can be used in combinations with the nanoparticles described herein where an ingredient of such compositions can optionally be replaced by a nanoparticle described herein, or a compound described herein can be added to the composition.

Useful dosages of the nanoparticles described herein can be determined by comparing their in vitro activity, and in vivo activity in animal models. Methods for the extrapolation of effective dosages in mice, and other animals, to humans are known to those in the art, particularly with the benefit of the present disclosure. In some aspects, the amount of a nanoparticle or a derivative thereof required for use in treatment will vary not only with the particular nanoparticle or salt selected but also with the route of administration, the nature of the condition being treated, and the age and condition of the patient, and will be ultimately at the discretion of an attendant physician or clinician.

The nanoparticle, or the composition thereof can be conveniently administered in a unit dosage form, for example, containing 5 to 1000 mg/m², conveniently 10 to 750 mg/m², most conveniently, 50 to 500 mg/m² of active ingredient per unit dosage form. The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

In some aspects, the nanoparticles may be used to deliver therapeutics to a subject in combination with any antioxidant and/or iron-chelating activity. For example, as discussed herein, the nanoparticle can be used to encapsulate a therapeutic agent within the core of the nanoparticle and can thus facilitate delivery of the therapeutic agent to a subject. In yet additional aspects, the nanoparticle can be used in an assay and/or medical imaging (e.g., a magnetic resonance imaging (MRI) contrast agent or a computed tomography (CT) contrast agent), such as by serving as conduit for a fluorescent marker or a contrast agent for medical imaging.

### Overview of Several Aspects

Disclosed herein are aspects of a nanoparticle comprising a plurality of polymers, wherein each polymer of the plurality of polymers independently has a structure according to Formula I:

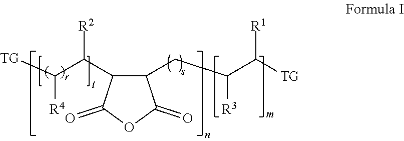

where m, n, and t, independently are integers ≥1; each TG independently is a terminating group; each R¹, R², R³, and R⁴, independently, for each occurrence, is H or comprises an aryl group; each r independently, for each occurrence, is 1 or 0; each s independently, for each occurrence, is 1 or 0; and provided that, (i) at least one R¹, R², R³, or R⁴, is not, or is other than, phenyl, (ii) one of R¹ or R³, for each occurrence, comprises the aryl group, and (iii) one of R² or R⁴, for each occurrence, comprises the aryl group.

In any or all of the above aspects, (i) each R¹ and R³, independently, are the same for each occurrence and at least one of R¹ or R³ is a phenol group, a dihydroxybenzene group, a trihydroxybenzene group, a tetrahydroxybenzene group, or a pentahydroxybenzene group and the other of R¹ or R³ is H; and (ii) each R² and R⁴, independently, are the same for each occurrence and at least one of R² or R⁴ is a phenol group, a dihydroxybenzene group, a trihydroxybenzene group, a tetrahydroxybenzene group, or a pentahydroxybenzene group and the other of R² or R⁴ is H.

In any or all of the above aspects, each R¹ and R³, independently, are the same for each occurrence and at least one of R¹ or R³ is a catechol group and the other of R¹ or R³ is H; (ii) each R² and R⁴, independently, are the same for each occurrence and at least one of R² or R⁴ is a catechol group and the other of R² or R⁴ is H; and (iii) one TG is

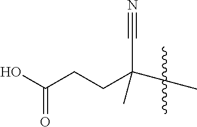 [SMILES: *C(C)(C#N)CCC(=O)O]

and the other TG is

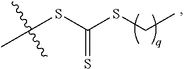

wherein q is an integer ranging from 0 to 20.

In any or all of the above aspects, each aryl group independently, for each occurrence, comprises a hydroxy group, an electron-withdrawing group, or a combination thereof.

In any or all of the above aspects, the plurality of polymers comprise poly(4-vinylcatechol-co-maleic anhydride)-block poly(4-vinylcatechol) (PCatMA-b-PCat) diblock copolymers, wherein each polymer comprises a poly(4-vinylcatechol-co-maleic anhydride) (PCatMA) block and a poly(4-vinylcatechol) (PCat) block.

In any or all of the above aspects, each polymer of the plurality of polymers independently has a structure according to Formula IA, Formula IA′, or Formula IA″:

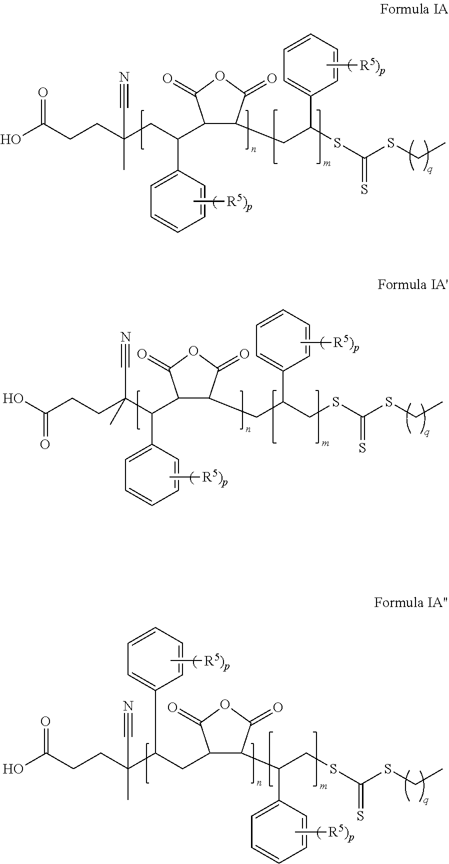

wherein each R⁵ independently, for each occurrence, is selected from —OH, a halogen, or a nitro group; each p, for each occurrence, is an integer selected from 0 to 5; and q is an integer selected from 0 to 20.

In any or all of the above aspects, each polymer of the plurality of polymers independently has a structure according to Formula IIB, IIB′, or IIB″:

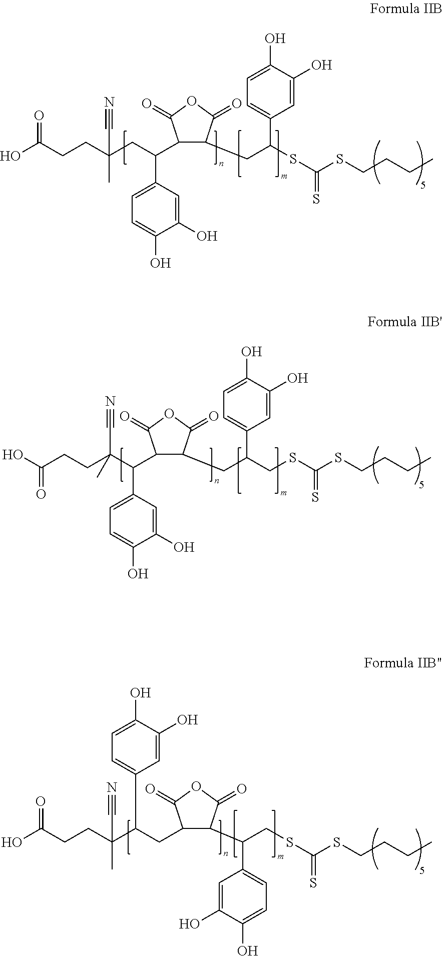

where m and n independently are integers ≥1.

In any or all of the above aspects, m is an integer ranging from 10 to 500; and n is an integer ranging from 10 to 500.

In any or all of the above aspects, the polymers have a number average molecular weight ranging from 10 kDa to 150 kDa, as determined by gel permeation chromatography or nuclear magnetic resonance spectroscopy calculation.

In any or all of the above aspects, a plurality of the nanoparticles has an average diameter of from 80 nm to 180 nm, as determined by dynamic light scattering.

In any or all of the above aspects, the polymers have a size distribution with a polydispersity index of from 1.0 to 1.6, as determined by dynamic light scattering.

In any or all of the above aspects, the nanoparticle has a micellar structure defining a core and wherein a therapeutic agent, a detectable label, or a combination thereof, is encapsulated in the core.

Also disclosed herein are aspects of a pharmaceutical composition comprising a nanoparticle according to any or all of the above aspects, and at least one pharmaceutically acceptable excipient.

Also disclosed herein are aspects of a method of synthesizing a nanoparticle, the method comprising: preparing a plurality of polymers; preparing a solution of the plurality of polymers in acetone; and dialyzing the solution against water, whereby polymers of the plurality of polymers form into nanoparticles, wherein each polymer of the plurality of polymers has a structure according to Formula I

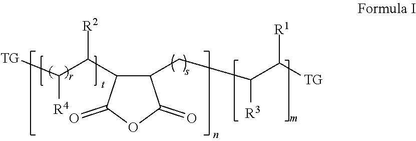

where m, n, and t, independently are integers ≥1; each TG independently is a terminating group; each R¹, R², R³, and R⁴, independently, for each occurrence, is H or comprises an aryl group; each r independently, for each occurrence, is 1 or 0; each s independently, for each occurrence, is 1 or 0; and provided that, (i) at least one R¹, R², R³, or R⁴, is not, or is other than, phenyl, (ii) one of R¹ or R³, for each occurrence, comprises the aryl group, and (iii) one of R² or R⁴, for each occurrence, comprises the aryl group.

In any or all of the above aspects, preparing the plurality of polymers comprises combining maleic anhydride and a vinyl aryl-containing monomer with a chain transfer agent, an initiator, and a solvent to form a monomer solution; and heating the monomer solution at a temperature effective to induce polymerization and produce the plurality of polymers.

In any or all of the above aspects, the method further comprises adding a therapeutic agent, a detectable label, or a combination thereof to the solution; and dialyzing the solution against water provides nanoparticles encapsulating the therapeutic agent, the detectable label, or the combination thereof.

Also disclosed herein are aspects of a method comprising administering a nanoparticle according to any or all of the above aspects, or a pharmaceutically acceptable composition thereof, to a subject having, or at risk of having, inflammation and/or iron-dependent cell death.

In any or all of the above aspects, the method further comprises contacting a bone cell with the nanoparticle.

In any or all of the above aspects, the subject has a bone fracture and the bone cell is at a site of the bone fracture.

In any or all of the above aspects, the nanoparticle scavenges reactive oxygen species and/or chelates iron.

### EXAMPLES

Aspects of the present teachings can be further understood in light of the following examples.

### Example 1—Statistical Methods

Statistics: Experimental sample sizes were informed by preliminary data and previous studies. Power analyses are conducted where applicable to ensure detection of biologically meaningful differences with α=0.05 and power ≥0.8. Data are represented as means±standard deviation unless otherwise noted. All data are compared for statistically significant differences between mean values using GraphPad Prism via one-way or two-way ANOVA and accompanied by Tukey's or Bonferroni's post hoc testing. Datasets are initially tested for normality using the Shapiro-Wilk test and for homogeneity of variance using Levene's test. If data normality cannot be assumed, then the non-parametric Kruskal-Wallis test is used to analyze differences. If Levene's test indicates significant data variance, Welch's ANOVA is performed to determine significant differences.

DOE: Model significance is assessed by lack-of-fit testing, adjusted R² values, and examination of residuals using JMP PRO. Significant main effects, interaction terms, and curvature are identified to optimize PCat NP composition.

Cell culture: To maintain rigor, the assessment and interpretation of ferroptosis experiments was conducted in accordance with international guidelines. All cell culture experiments had at least three technical and three independent biological replicates. Iron-containing media were prepared fresh each time to mitigate the potential formation of insoluble iron hydroxides.

In vivo: Due to the small n in these examples, the experiments are not designed to detect powered sex-related differences. Despite this, equal numbers of male and female mice can be incorporated to consider the influence of sex-based hormones, as they may influence biodistribution and fracture healing.

### Example 2—Synthesis of poly(4-vinylcatechol-co-maleic anhydride)-block-poly(4-vinylcatechol) (PCatMA-b-PCat) diblock copolymers

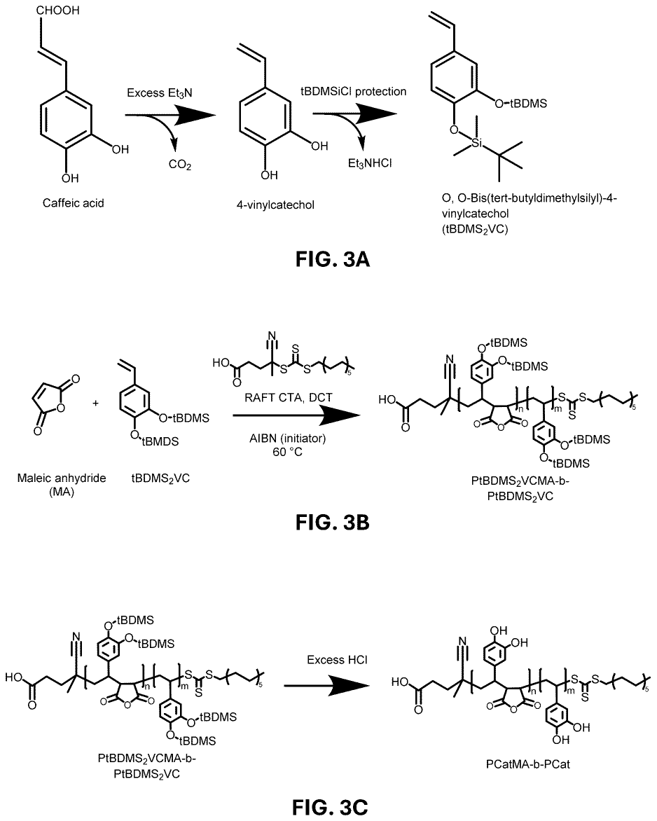

Vinylcatechol (VC) monomers were synthesized from a caffeic acid precursor and protected with tert-butyldimethylsilyl ether (tBDMS) groups to form 0,0-bis(tert-butydimethylsilyl)-4-vinylcatechol (tBDMS₂VC) in a one-pot reaction. (FIG. 3A). tBDMS₂VC allows polymerization without the risk of polyphenolic H abstraction.

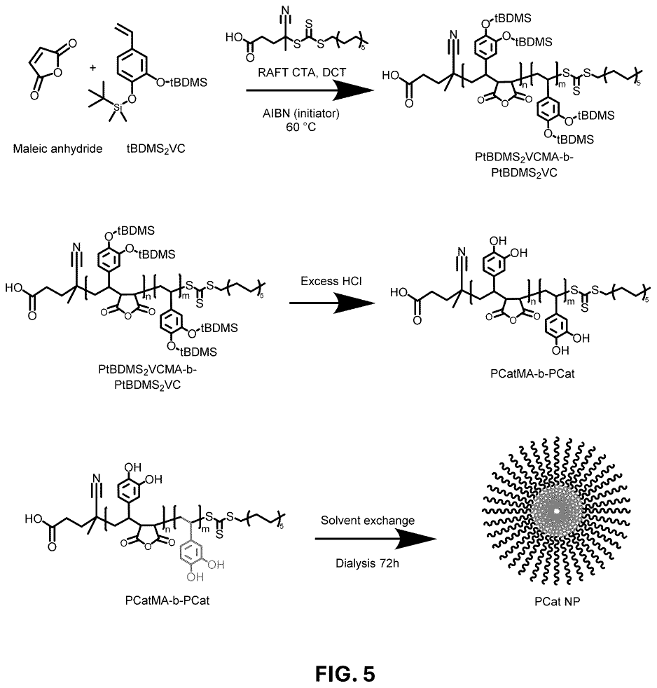

Amphiphilic poly(0,0-bis(tert-butyldimethylsilyl)-4-vinylcatechol-co-maleic anhydride)-block-poly(0,0-bis(tert-butyldimethylsilyl)-4-vinylcatechol) (ptBDMS₂VCMA-b-ptBDMS₂VC) diblock copolymers were synthesized via reversible addition-fragmentation chain transfer (RAFT) polymerization by dissolving tBDMS₂VC (>98%, synthesized), maleic anhydride (>99%, ACS grade) and 4-cyano-4-[(dodecylsulfanylthiocarbonyl) sulfanyl]pentanoic acid (DCT) (>98%, synthesized) ([tBDMS₂VC]:[maleic anhydride]:[DCT]=300:100:1) in 1,4-dioxane (28% w/w). (FIGS. 3B-3C and FIG. 5). While toluene works well as a solvent for the homopolymerization of tBDMS₂VC, copolymerizations with maleic anhydride (MA) require dioxane due to its poor solubility in nonpolar solvents.

AIBN was recrystallized from methanol and used as an initiator by addition to the reaction solution ([DCT]:[AIBN]=10:1). The DCT was the RAFT chain transfer agent.

1,2,3,4-tetrahydronapththalene was used as a reference molecule to track polymer conversion, via incorporation in 1,4-dioxane (5% v/v). This reaction mixture was stirred and purged with nitrogen gas over ice for 45 minutes to conduct polymerization in an inert atmosphere free from oxygen inhibition. Before the polymerization was initiated, a small fraction approximately 0.1 mL in volume was taken for NMR analysis in deuterated chloroform. The reagent solution was then placed in a 60° C. oil bath for 72 hours before freezing at −78° C. in dry ice and exposing to air for termination without loss of solvent or reference molecules. The polymer solution was then sealed and allowed to come to room temperature before taking a final aliquot of approximately 0.1 mL for post-polymerization NMR analysis in deuterated chloroform. Monomer conversion was calculated by comparing the integral areas of vinylic proton peaks prior to and following polymerization using NMR. The remaining polymer solution was further diluted in pentanes before dropwise addition and subsequent precipitation in chilled acetonitrile (−20° C.). The precipitated product was then dried in a vacuum before characterization of molecular weight and polydispersity index using GPC (see Table 1).

In some examples, measured molecular weight was considerably lower than the calculated values from NMR analysis. The calculated values from the NMR analysis were based on the assumption that the polymerization proceeds in a controlled fashion inherent to RAFT polymerization, leading to a synthesized polymer with a low PDI index (<1.2). Disproportionate termination was hypothesized to have been occurring using a 5:1 [DCT]:[AIBN] ratio, skewing the number average molecular weight lower and increasing the PDI index. In subsequent replicates, the ratio of the chain transfer agent to initiator was increased to 10:1 [DCT]:[AIBN], which gave evidence of successfully controlled polymerization given the low PDI indices and molecular weights closer to calculated values.

TABLE 1
Preliminary polymerization results.

| | | | MA | Block One | Block Two | Diblock Copolymer | | |
|---|---|---|---|---|---|---|---|---|
| | | | conv | Calculated | Calculated | Calculated | Measured | |
| Polymer | [MA]₀ | [tBMDS₂VC]₀ | (%) | Mₙ (kDa) | Mₙ (kDa) | Mₙ (kDa) | Mₙ (kDa) | M_{w}/Mₙ |
| One-pot | 0.49 | 1.45 | 100 | n/a | 46.2 | n/a | 32.2 | 1.45 |
| 3 | | | | | | | | |
| Two-pot | n/a | 0.26 | n/a | 51 | 32.2 | 60.8 | 34.3 | 1.57 |
| 3.1 | | | | | | | | |
| One-pot | 0.34 | 1.04 | 100 | 55 | 46.2 | 69.9 | 22.5 | 1.57 |
| 4 | | | | | | | | |

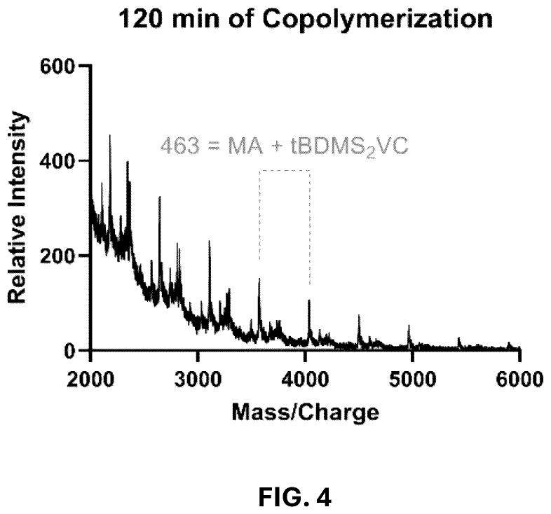

Due to the complementary electronic properties of MA and tBDMS₂VC, copolymerization may proceed in an alternating fashion similar to the case of MA and styrene copolymerization. Evidence of alternating copolymerization was seen in the 120-minute reaction time, indicated by the difference between mass peaks being equivalent to the sum of the molecular weights of MA and tBDMS₂VC (see FIG. 4).

To expose catechol moieties and remove tert-butyldimethylsilyl ethers, protected tBDMS₂VC-containing polymers were dissolved in 1M HCl dioxane solution at 10 mg/mL. The reaction mixture was purged with argon gas over an ice bath for 20 minutes before refluxing at 80° C. over an oil bath for 48 hours. The reaction solution was then cooled to room temperature before concentrating down via rotary evaporation. The subsequent poly(4-vinylcatechol-co-maleic anhydride)-block-poly(4-vinylcatechol) (PCatMA-b-PCat) concentrate was then washed 3 times in chilled diethyl ether before drying under vacuum. The dried PCatMA-b-PCat pellet was then dissolved in deuterated dimethylsulfoxide for analysis via NMR, confirming the absence of protons associated with tert-butyldimethylsilyl ether protecting groups.

While NMR verified the success of the deprotection reaction, the PCatMA-b-PCat diblocks coordinated with trace heavy metals during NaHCO₃ purification, forming a dark-colored supramolecular metal-phenolic network that was unusable for moving forward with in vitro tests.

Using SEPLITE high-affinity ion exchange chelating resin beads to remove trace heavy metals from NaHCO₃ successfully isolated PCatMA-b-PCat copolymers after deprotection. PCatMA-b-PCat NPs form spontaneously when a solution of the polymer in acetone is dialyzed against deionized water as demonstrated by preliminary DLS results (see Table 2).

TABLE 2
Representative polymer and nanoparticles characterized
by ¹H-NMR, GPC, and DLS. DLS data are represented as
mean ± standard deviation with sample size n = 3.

| MA | | | | | |
|---|---|---|---|---|---|
| conver- | tBMDS₂VC | | | Block One | Block Two |
| sion | conversion | | tBMDS₂VC | Calculated | Calculated |
| (%) | (%) | MADP | DP | Mn (kDa) | Mn (kDa) |
| 100 | 52 | 97 | 156 | 45 | 22 |

| Diblock Copolymer | | | NP Assembly | | |
|---|---|---|---|---|---|
| Calculated Mn | Measured Mn | | Size | Zeta | |
| (kDa) | (kDa) | Mw · Mn | (nm) | (mV) | PDI |
| 67 | 65 | 1.09 | 130 ± 1 | −38 ± 1 | 0.3 |

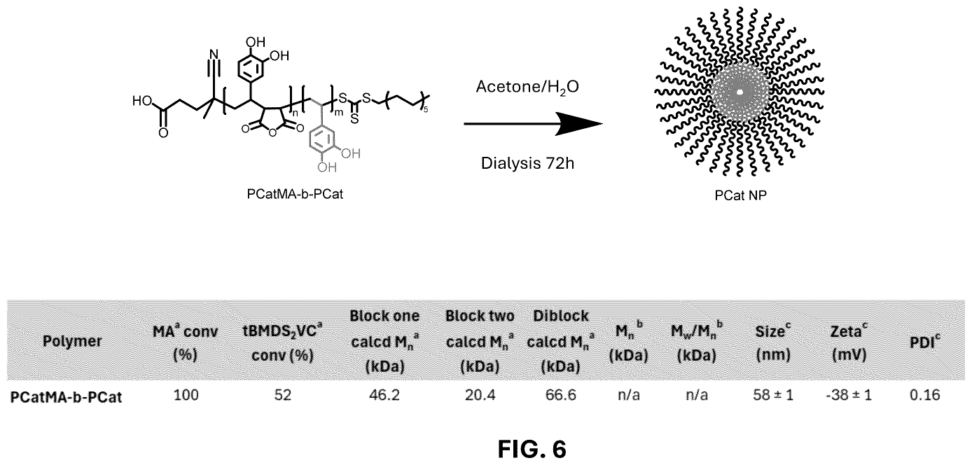

PCatMA-b-PCat diblock copolymers were self-assembled into nanoparticles via solvent exchange from dimethylsulfoxide (or acetone or dimethylformamide) to water via dropwise addition of polymer organic solution to water in a 1:1 volume ratio of organic phase to aqueous phase during constant stirring. The resulting suspension was then dialyzed against ultrapure MILLIQ water with a 10 kDa molecular weight cutoff (MWCO) dialysis tubing chilled and protected from light for 72 hours (see FIGS. 5 and 6).

NPs were characterized by using dynamic light scattering (DLS) and NP tracking analysis (NTA), and transmission electron microscopy (TEM), to determine size, zeta potential, and PDI. The design of experiments (DOE) tool in the JMP Pro statistical discovery platform was used to build a central composite design response surface model to identify how polymeric properties such as molar % catechol, block 1: block 2 molecular weight ratios, and total molecular weight impact critical NP outcomes such as iron chelation, antioxidant capacity, and cytocompatibility (Table 3).

TABLE 3
Inputs and responses for the central composite design model
in Aim 1A. Ranges were determined from preliminary data
and previous studies on what is experimentally possible.

| Polymeric Inputs | Range | Responses |
|---|---|---|
| Mol % catechol | 20-60 | Iron chelation capacity, |
| Block 1: Block 2 Mₙ ratio | 0.75-2.0 | antioxidant capacity, & |
| Total Mₙ (kDa) | 20-40 | cytocompatibility |

Successful RAFT polymerization was validated by a molecular weight PDI index (M_{w}/Mₙ)<1.2, demonstrating a monodisperse polymer population. Synthesized polymers maintain catechol functionality during assembly and storage before NP treatments, as verified by the percentage of retained phenolic protons using spectroscopic analysis. For in vitro and in vivo studies, PCat NPs were 50-120 nm in size, have zeta potential <−20 mV, and PDI <0.3, which may be favorable for fracture accumulation by the ability of NPs to extravasate through leaky vasculature at inflamed injury sites (termed the ‘ELVIS’ effect), and may result in little to no off-target biodistribution in vivo.

DLS results show the successful assembly of polycatecholic NPs with anionic surface charge. (Table 2).

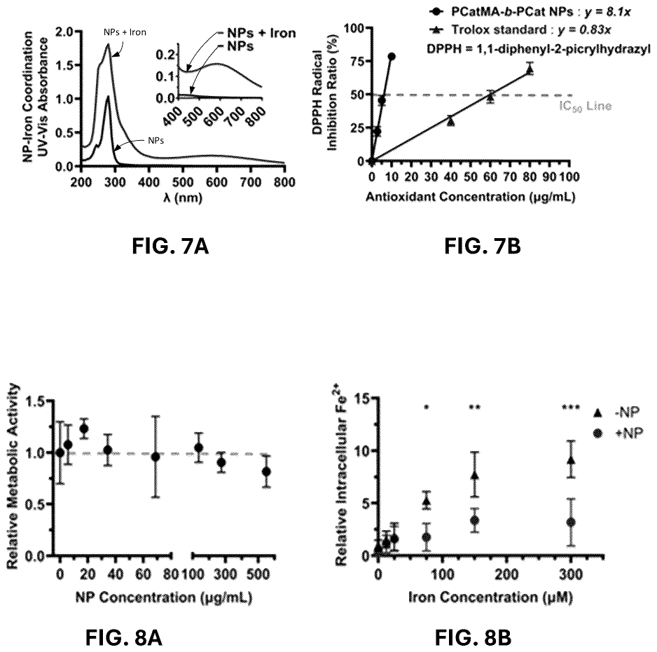

The ability for PCat NPs to chelate iron was assessed using UV-vis spectroscopy. PCat NPs were prepared at 0.0005 w/v % in Tris buffer at pH 7.4. The resulting suspension was split evenly for UV-vis spectroscopic analysis. To one vial, ferric citrate was added to a final concentration of 0.5 mM ferric iron. The resulting suspension changed color to blue-gray following addition, characteristic of ferric catecholate coordination complex formation. To the other vial, a volumetric equivalent of water was added to maintain the same dilution of PCat NPs. The two vials were characterized for their absorbance spectra from 200-800 nm confirming the presence of a broad absorbance peak from 450-800 nm in the iron-treated PCat NP vial, characteristic of ferric catecholate coordination complexation (see FIG. 7A). This confirmed the ability of PCat NPs to chelate ferric iron at physiological pH.

The antioxidant capacity of PCat NPs compared to a Vitamin E (TROLOX) standard was assessed using a 1,1-diphenyl-2-picrylhydrazl (DPPH) radical scavenging assay. Preliminary experiments verified the linear concentration range for DPPH radical scavenging was between 2 and 10 μg/mL for PCat NPs. Concentrations were tested at 2.5, 5, and 10 μg/mL and fitted with a linear regression line (R²=0.99, n=3). The resulting UV-vis absorbance results were then converted to % DPPH radical inhibition and plotted against antioxidant concentration to determine half-maximal inhibitory concentrations (IC₅₀). The antioxidant capacity is then determined by comparing the IC₅₀ to a known antioxidant Vitamin E analog standard, TROLOX. The calculation to determine Trolox equivalent antioxidant capacity (TEAC) is as follows, TEAC=IC50_{Trolox}/IC50_{PCat}. The PCat NPs exhibited an antioxidant capacity approximately 9.7 times that of the TROLOX vitamin E analog (FIG. 7B) indicating their high antioxidant potential.

Mol % catechol functionality was altered by substituting styrene monomers in place of tBDMS₂VC monomers. Molecular weight ratios were altered through changing the monomer feed or monomer to chain transfer agent ratios. Cytocompatibility was assessed using ALAMARBLUE metabolic activity and lactate dehydrogenase live/dead assays to determine NP IC₅₀ concentrations on RAW 264.7 murine-derived macrophages and MC3T3-E1 murine-derived pre-osteoblasts. The three to five best-performing PCat copolymer formulations identified from the response surface model were advanced for further in vitro and in vivo testing. Using the best PCat performers, NP assembly conditions were empirically tested to find methods that result in the desired size, zeta, and PDI.

### Example 3—Determination of Anti-Ferroptotic Efficacy of PCat NPs In Vitro

Macrophages are immune cells critical for orchestrating bone regeneration and iron regulation in fractures. Polarization of macrophages from pro-inflammatory to pro-regenerative phenotypes is hindered in aged fractures, impairing fracture healing. Age-related iron accumulation leads to sustained pro-inflammatory macrophage polarization, thereby elevating concentrations of inflammatory cytokines that inhibit osteogenesis and promote paracrine-mediated ferroptotic death in osteoblasts (see FIG. 1). Due to their high phagocytic activity and prevalence during the early stages of fracture healing, macrophages may be the main cells that uptake PCat NPs in vivo, as demonstrated in previous studies. PCat NPs may polarize macrophages to pro-regenerative phenotypes in age-related iron overload models and subsequently mitigate osteoblast ferroptosis.

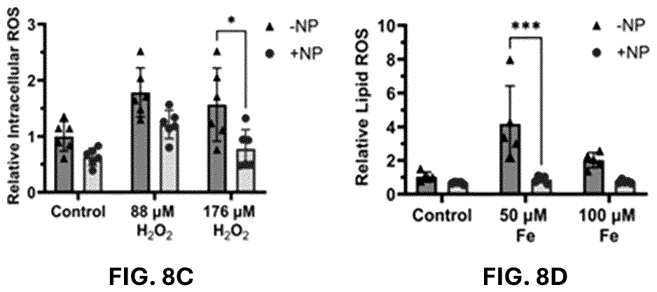

Preliminary data suggest that PCat NPs have promising anti-ferroptotic potential in vitro. To test the cytocompatibility of PCat NPs in pre-osteoblasts, an ALAMARBLUE metabolic assay was performed after PCat NP treatments (FIG. 8A). ALAMARBLUE metabolic assay performed after treating MC3T3-E1 cells with NP concentrations up to 550 μg/mL. No significant effect on metabolic activity was measured, indicating that PCat NPs are cytocompatible in pre-osteoblasts. To test the iron chelation capabilities of PCat NPs in vitro, pre-osteoblasts were incubated in ferrous iron-rich conditions before PCat NP treatment. MC3T3-E1s were incubated with ferrous sulfate heptahydrate concentrations ranging from 0 UM to 300 UM for 30 minutes before PCat NP treatment. A ferrous-specific FERROORANGE fluorescent probe was used to determine relative intracellular iron content to untreated controls. Intracellular ferrous iron content was significantly reduced in the groups treated with 75 μM iron and above (FIG. 8B), indicating PCat NPs chelate iron in vitro. This is likely due to the anti-Fenton potential of PCat NPs by removing ferric ions from the cytosolic iron pool (FIG. 2B). To test the ROS scavenging capability of PCat NPs, pre-osteoblasts were incubated with the redox fluorescent probe diacetyldichlorofluorescein (DCFH-DA) with and without PCat NP treatment before incubation in ROS-rich H₂O₂ conditions (FIG. 8C). MC3T3-E1s were incubated for 30 minutes with the redox fluorescent probe DCFH-DA before PCat NP treatment and incubation in H₂O₂ conditions to determine relative intracellular ROS to the untreated control. PCat NP treatment significantly reduced intracellular ROS in the 176 μM H₂O₂ group, indicating PCat NP antioxidant activity is retained in vitro. The ability of PCat NPs to reduce lipid peroxides was tested using a BDP 581/591 C11 fluorescent probe (FIG. 8D). Following incubation with ferrous iron, MC3T3-E1s were incubated with a BDP 581/591 C11 fluorescent probe and PCat NPs for 30 minutes to determine relative lipid peroxidation compared to the untreated control. A PCat NP concentration of 60 μg/mL was used in all studies. Relative intracellular lipid peroxide levels decreased after PCat NP treatments, suggesting that PCat NPs can reduce lipid peroxidation in vitro, further supporting their anti-ferroptotic potential.

NP cytocompatibility was also assessed in RAW 264.7 macrophages using lactate dehydrogenase (LDH) and ALAMARBLUE assays. The functions of PCat NPs in macrophages, namely scavenging intracellular ROS and chelating cytosolic iron, were measured the using DCFH-DA and FERROORANGE, respectively. PICOGREEN assays were performed to normalize results to DNA content.

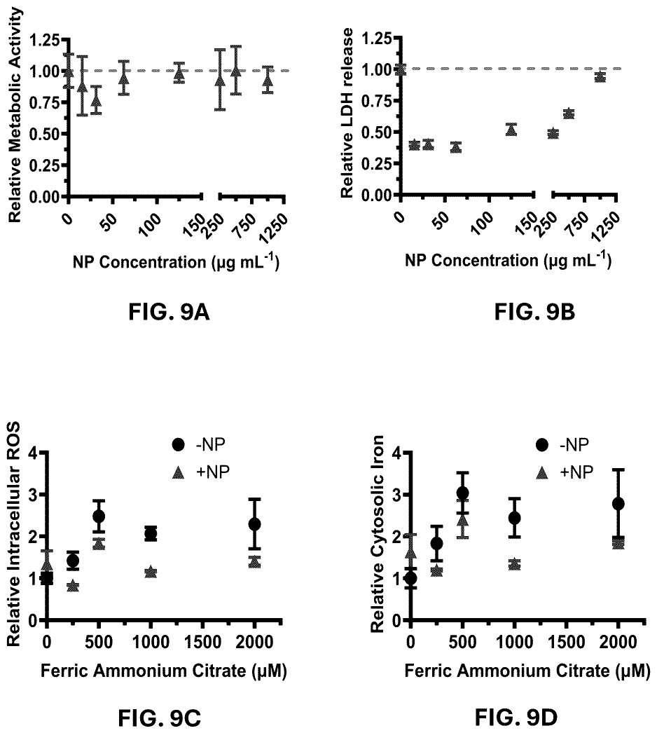

PCat NPs were cytocompatibile, with no measurable reduction in metabolic activity up to 1000 μg/mL (FIG. 9A). LDH release decreased after PCat NP treatment at concentrations lower than 1000 μg/mL, suggesting a possible change in metabolism from glycolysis to oxidative phosphorylation (FIG. 9B). PCat NPs (tested at a concentration of 125 μg/mL) show potential in scavenging intracellular ROS (FIG. 9C) and chelating cytosolic iron (FIG. 9D) as indicated by the decrease in intracellular ROS and cytosolic iron following NP treatment in iron-overloaded macrophages. This example suggests that PCat NPs may be powerful therapeutics for promoting pro-regenerative macrophage polarization in settings of chronic inflammation, including due to age.

PCat NPs may promote pro-regenerative macrophage polarization. This can include changes in gene expression, surface marker presentation, and metabolic pathways as indicators of macrophage phenotypic changes. The biodistribution and effect of PCat NPs in promoting healing can be evaluated in aged murine fracture models.

### Example 4—Determining the Efficacy of PCat NPs in Promoting the Polarization of Macrophages to a Pro-Regenerative Phenotype

RAW 264.7 macrophages are used to study the bioactivity of PCat NPs. Pro-inflammatory macrophages can be differentiated through 1) classical activation via treatment with LPS and interferon-γ (M1_{LPS}) for 48 h, and 2) excess iron treatment with ferric ammonium citrate (FAC) for 48 hours (M1_{FAC}). As positive controls for pro-regenerative polarization, macrophages are treated with interleukin-4 for 48 hours (M2). The polyphenol antioxidant quercetin and iron chelator deferoxamine can be used as free drug control due to previous literature showing their effect on macrophage pro-regenerative polarization. A range of PCat NP concentrations can be tested to evaluate their efficacy in pro-regenerative macrophage polarization. Intracellular ROS and cytosolic iron are measured by comparing relative fluorescence intensities to M2 controls using DCFH-DA and FERROORANGE fluorescent probes (as in Example 3), respectively. At timepoints 1 day, 4 days, and 7 days post-treatment, macrophages can be further characterized via cytokine levels via enzyme-linked immunosorbent assays (ELISA), by gene expression via quantitative real-time polymerase chain reaction (qPCR), and cell surface markers via flow cytometry.

This example can elucidate the efficacy of PCat NPs in promoting pro-regenerative macrophage polarization in iron overload conditions. Metrics include significant reductions in intracellular iron and ROS in M1 macrophages after PCat NP treatment. PCat NP treatment concentrations for subsequent experiments can be determined by the decrease in pro-inflammatory markers relative to M1 controls and increase in pro-regenerative potential measured by ELISA, qPCR, and flow cytometry.

If PCat NPs do not polarize RAW 264.7 cells to a pro-regenerative phenotype, the effects of PCat NPs on bone-marrow-derived macrophages (BMDMs) can be evaluated before testing new hypotheses, as subtle differences in phagocytic and metabolic function have been documented between BMDMs and RAW cells.

### Example 5—Determination of Therapeutic Efficacy of PCat NPs in Promoting Osteogenic Potential in Iron Overload In Vitro Using Coculture Models

A conditioned media co-culture model can be employed to study the effects of paracrine signaling between PCat NP-treated RAW 264.7 macrophages and MC3T3-E1 pre-osteoblasts. Macrophages are differentiated as described Example 4, 48 hours before being treated with PCat NPs or free drug conditions. MC3T3-E1s can then be seeded separately in osteogenic media. As positive ferroptosis controls, MC3T3-E1s can be cultured with conditioned media from pro-inflammatory macrophages (M1_{LPS} or M1_{FAC}). As pro-regenerative controls, MC3T3-E1s can be cultured with conditioned media from M2 macrophages. As baseline control, MC3T3-E1 monocultures can undergo the same experimental analysis without conditioned media. Studies can be conducted over 21 days to evaluate osteogenic activity over a biologically relevant period. To evaluate ferroptosis in MC3T3-E1s, intracellular ROS and iron are measured as described in Example 4 using fluorescent confocal microscopy, and lipid peroxidation levels are measured using malondialdehyde (MDA) biochemical assays (days 1, 4, 7, and 14). Relevant ferroptosis and osteogenic markers expressed in MC3T3-E1s (days 1, 4, 7, and 14) can be quantified using ELISA and qPCR. Furthermore, alkaline phosphatase (ALP) staining (day 7) and alizarin red calcium deposition staining (day 14) can be performed on MC3T3-E1s as measures of osteogenic activity. To investigate the influence of age-related iron overload on pre-osteoblasts, experiments can be performed with and without 24 hour FAC pretreatment on MC3T3-E1s.

Overall, this example evaluates the effect of PCat NP-treated macrophages mitigating paracrine-mediated ferroptosis induced in osteoblasts in iron overload models. Successful reversal of ferroptosis can be determined through significant reduction in intracellular ROS, cytosolic iron, lipid peroxidation, and an increase in anti-ferroptotic genetic markers. Furthermore, restoration of osteogenic potential is determined by a significant increase in osteogenic markers and calcium deposition.

### Example 6—Determination of Therapeutic Efficacy of PCat NPs to Promote Age-Related Fracture Healing In Vivo

Aging is associated with impaired fracture healing through chronic inflammation, oxidative damage, and delayed endochondral ossification. While growing evidence suggests a combinatorial therapeutic approach of iron chelation and polyphenolic antioxidants promotes regeneration in osteoporotic fractures, the efficacy in healing general age-related fractures remains unknown despite iron accumulation and oxidative stress underlying both conditions. PCat NPs may prove a promising minimally invasive therapeutic strategy in promoting age-related fracture healing due to their high antioxidant capacity, iron chelation, and ELVIS-mediated fracture accumulation. Furthermore, PCat NPs may be biocompatible at therapeutically relevant doses.

MDA content was measured in undecalcified bone tissue from 11 women. Age-related osteoporotic hip fractures showed increased lipid peroxidation markers and decreased glutathione peroxidase 4 (GPX4) as a function of serum ferritin levels >150 μg/L in human bone tissue. This supports recent findings that age-related iron accumulation in bone tissue results in ferroptotic death of osteoblasts, indicated by the depletion of GPX4, the key antioxidant enzyme that prevents lipid peroxide accumulation. Separately, polyphenolic antioxidant treatments were used to treat fractures in ovariectomy-induced (Ovx) osteoporotic rats. Treatments with icariin, a polyphenolic flavonoid, significantly improved bone mineral density and restored GPX4 levels in osteoporotic fractures, indicating the promising anti-ferroptotic potential. In some aspects, treatments required high doses of drugs (50 mg/kg and 150 mg/kg for low (LICA) and high (HICA) doses, respectively) administered daily through intragastric gavage due to the poor accumulation of small-molecule drugs at fractures.

### Example 7—Biodistribution and Biocompatibility of PCat NPs In Vivo

A mid-diaphyseal femur fracture model is employed using 18-20 month old C57BL/6 mice, as they can model age-related impaired fracture healing. Live animal imaging analyzes biodistribution of IR780 near-infrared loaded PCat NPs injected retro-orbitally 3 days post-surgery (bone and off-target organs: liver, spleen, kidneys, lungs, heart, etc.) until fluorescence is undetectable (Table 4). In tandem with biodistribution studies, long-term systemic side effects of PCat NPs vs. placebo (PBS) and free-drug treatments are evaluated by collecting submandibular bleeds at days 0, 1, 7, 14, and 28 after NP treatment to isolate serum and analyze toxicity-related enzymes (i.e., AST and ALT for liver toxicity) using commercially available assay kits. Additionally, serum ferritin and MDA levels can be measured as markers of iron overload and oxidative stress.

TABLE 4
In vivo experimental group design for Aim 3

| Experiment | Time points | | Groups | n/group | Total n |
|---|---|---|---|---|---|
| Biodistribution/ | Longitudinal | | 3 (placebo, PCat | 6 | 18 |
| Serum | | | NP, free drug) | | |
| Enzymes | | | | | |
| Histology | 2, 7, 14, 28 | d | 3 (placebo, PCat | 4 | 48 |
| | | | NP, free drug) | | |
| μCT | 14, 28 | d | 3 (placebo, PCat | 6 | 36 |
| | | | NP, free drug) | | |
| Biomechanics | 28 | d | 3 (placebo, PCat | 8 | 24 |
| | | | NP, free drug) | | |

PCat NP fracture accumulation and retention is determined by increasing fluorescent intensity from hour 4 and onward until a maximum is reached. The percent fracture accumulation can be determined by comparing IR780 fluorescent intensity with off-target organs. As a measure of biocompatibility, treated mice may show no toxic systemic side effects from serum studies, no reduction in weight, and no behavioral changes associated with pain or lethargy compared to placebo. A reduction in MDA and ferritin levels after PCat NP or free-drug treatments can serve as preliminary evidence of anti-ferroptotic potential in vivo.

### Example 8—Determination of Therapeutic Efficacy of PCat NPs to Promote Fracture Healing in Aged Mice

Using the in vivo model of Example 7, treatments (saline, free drug, and PCat NP) are injected retro-orbitally into mice 3 days post-surgery. Fracture healing and callus formation can be evaluated using micro-computed tomography (μCT) 2- and 4-weeks post-treatment to analyze bone volume and bone mineral density, which indicate restored bone quality post age-related fracture (Table 3). Additionally, fractures can be excised to quantify torsional strength, with x-ray imaging conducted after failure to investigate fracture modes (Table 4). Histology can be performed on harvested tissues using orange G/alcian blue and hematoxylin/eosin to monitor successful fracture healing (Table 4). Perl's Prussian Blue and MDA staining can be performed to assess tissue iron and lipid peroxidation content. Macrophage phenotypic ratio in fractures can be quantified through immunofluorescence to determine the effect of treatments on restoring regenerative populations.

Metrics of successful fracture healing include enhanced callus formation in PCat versus placebo, which are verifiable using μCT and histology. Reductions in iron and MDA staining can be interpreted as successful mitigation of age-related iron accumulation and ferroptosis. Torsional strength studies can be used to show the therapeutic efficacy of PCat NPs in promoting age-related fracture healing. An increase in pro-regenerative macrophages verified through immunofluorescence can demonstrate the efficacy of PCat NPs to attenuate the sustained inflammatory polarization of macrophages in vivo.

If PCat NPs fail to accumulate in fractures, then a bone-targeting peptide (TBP) can be conjugated to the corona of PCat NPs through the maleic anhydride functionality. If TBP-PCat NPs still fail to accumulate at the fracture site via systemic injection, localized injection methods can be employed to evaluate the therapeutic potential of PCat NPs.

### Example 9—Incorporation of Styrene Monomers

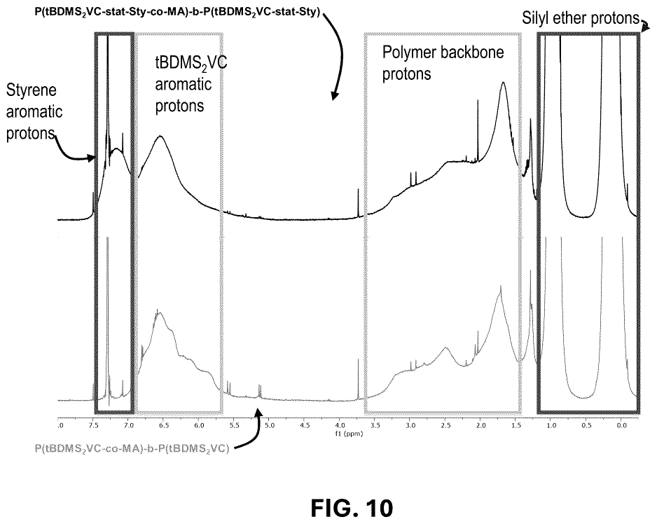

Diblock terpolymers were synthesized to include various molar amounts of styrene, maleic anhydride, and tBDMS₂VC. In some example, this was achieved by adjusting the molar ratio of styrene to tBDMS₂VC during the synthesis procedure, resulting in poly(styrene-stat-tBDMS₂VC-co-maleic anhydride)-block-poly(styrene-stat-tBDMS₂VC) diblock terpolymers with statistically random incorporation of styrene and tBDMS₂VC. In other aspects, a two-step synthesis can be used via random incorporation or segregation of either styrene or tBDMS₂VC in either block resulting in poly(tBDMS₂VC-co-maleic anhydride)-block-poly(styrene), poly(styrene-alt-maleic anhydride)-block-poly(tBDMS₂VC), poly(styrene-stat-tBDMS₂VC-co-maleic anhydride)-block-poly(tBDMS₂VC), or poly(tBDMS₂VC-co-maleic anhydride)-block-poly(styrene-stat-tBDMS₂VC). For the two-step synthesis method, the first block can be synthesized following any combination of [styrene]:[tBDMS₂VC]:[maleic anhydride] where [styrene]+ [tBDMS₂VC]=[maleic anhydride]. After polymerization, the first block can be isolated following the precipitation and drying method detailed above, before dissolving the synthesized block in dioxane as a macro-chain transfer agent and reinitiating the polymerization of the second block in the presence of any combination of styrene and/or tBDMS₂VC monomers with AIBN as the initiator. The NMR spectra of FIG. 10 show incorporation of styrene in a statistically random fashion during a one-pot synthesis method.

### Example 10—PCat NP IR780 Loading Efficiency and Capacity

The ability of PCat NPs to load drugs was assessed using IR780, a fluorophore that functions as a model hydrophobic drug. IR780 is solubilized in acetone and loaded into PCat NPs. Residual acetone is removed via rotary evaporation before characterizing NPs. To characterize loading efficiency and capacity, 100 μL of IR780-loaded PCat NPs were added to a solution of 90% acetonitrile and 10% water to a volume of 1 mL in a microcentrifuge tube and centrifuged at 10,000 relative centrifugal force (RCF) for 30 minutes before analyzing 100 UL of the supernatant using UV-vis to calculate the concentration of loaded and unloaded IR780. Next, to determine free IR780, 100 L of IR780-loaded PCat NPs were added to an ultracentrifugation filtration tube and spun at 10,000 RCF for 30 minutes. The filtrate was collected and diluted 1:10 in 90% acetonitrile and 10% water to calculate unloaded IR780 concentration.

Table 5 shows properties of PCat NPs after IR780 loading. Size, PDI, and Zeta potential were assessed by DLS. Values in Table 5 are reported as mean+SD (n=3). Loading was characterized by UV-vis spectroscopy at 780 nm.

TABLE 5
Properties of PCat NPs after IR780 loading.

| | Size | Zeta | | Loading | Loading |
|---|---|---|---|---|---|
| Sample | (nm) | (mV) | PDI | efficiency (%) | capacity (%) |
| PCat NP | 117 ± 1 | −32 ± 1 | 0.23 | n/a | n/a |
| PCat NP_{IR780} | 135 ± 8 | −46 ± 2 | 0.31 | 99.9 | 11.6 |

### Example 11—Flow Cytometry Uptake

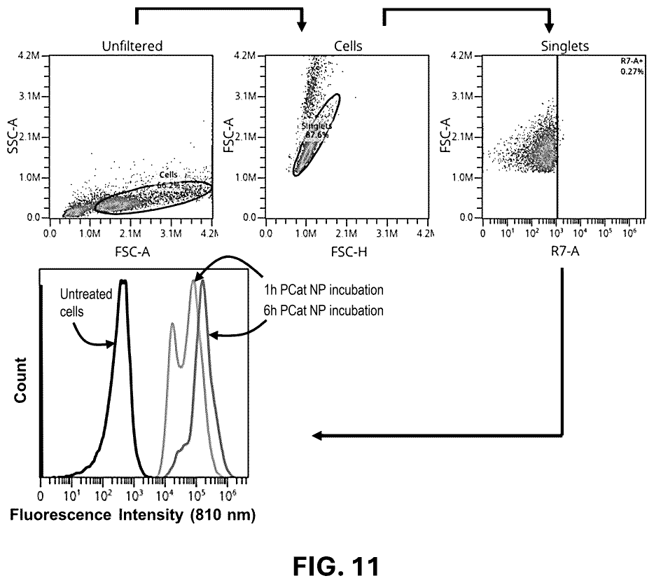

To assess the ability of macrophages to uptake PCat NPs, RAW 264.7 macrophages were treated with IR780-loaded PCat NPs at 60 μg/mL. RAW 264.7 macrophages were seeded at a density of 10,000 cells/cm² in a treated polystyrene six-well plate and allowed to adhere overnight at 5% CO₂ and 37° C. A fresh media change was performed at 24 hours. On the third day of incubation, one group went untreated as a control, another was incubated with IR780-loaded PCat NPs for six hours, and another with IR780-loaded PCat NPs for one hour. Following the PCat NP incubation time, cells were removed via cell scraping into respective tubes before centrifuging at 300 RCF for 5 minutes. The supernatant was discarded and pellets were washed with sterile 1×PBS twice before suspending in 300 μL of fluorescence-activated cell sorting (FACS) buffer. Cell suspensions were stored over ice and protected from light before immediate analysis using flow cytometry to assess the percent of IR780-positive cells. Flow cytometry analysis confirms 100% of RAW 264.7 cells were positive for IR780 signal after one hour and six hours of incubation time, indicating PCat NPs are readily endocytosed by RAW 264.7 cells (FIG. 11).

In view of the many possible aspects to which the principles of the disclosure may be applied, it should be recognized that the illustrated aspects are only preferred examples of the disclosure and should not be taken as limiting the scope of the disclosure. Rather, the scope is defined by the following claims. We therefore claim as our invention all that comes within the scope and spirit of these claims.

We claim:

1. A nanoparticle comprising a plurality of polymers, wherein each polymer of the plurality of polymers independently has a structure according to Formula I: 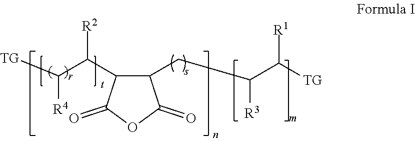
where m, n, and t, independently are integers ≥1;
each TG independently is a terminating group;
each R¹, R², R³, and R⁴, independently, for each occurrence, is H or comprises an aryl group;
each r independently, for each occurrence, is 1 or 0;
each s independently, for each occurrence, is 1 or 0; and
provided that,
(i) at least one R¹, R², R³, or R⁴, is not, or is other than, phenyl,
(ii) one of R¹ or R³, for each occurrence, comprises the aryl group, and
(iii) one of R² or R⁴, for each occurrence, comprises the aryl group.

2. The nanoparticle of claim 1, wherein (i) each R¹ and R³, independently, are the same for each occurrence and at least one of R¹ or R³ is a phenol group, a dihydroxybenzene group, a trihydroxybenzene group, a tetrahydroxybenzene group, or a pentahydroxybenzene group and the other of R¹ or R³ is H; and (ii) each R² and R⁴, independently, are the same for each occurrence and at least one of R² or R⁴ is a phenol group, a dihydroxybenzene group, a trihydroxybenzene group, a tetrahydroxybenzene group, or a pentahydroxybenzene group and the other of R² or R⁴ is H.

3. The nanoparticle of claim 2, wherein (i) each R¹ and R³, independently, are the same for each occurrence and at least one of R¹ or R³ is a catechol group and the other of R¹ or R³ is H; (ii) each R² and R⁴, independently, are the same for each occurrence and at least one of R² or R⁴ is a catechol group and the other of R² or R⁴ is H; and (iii) one TG is 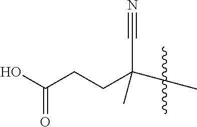 [SMILES: *C(C)(C#N)CCC(=O)O]
and the other TG is 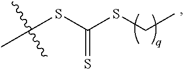
wherein q is an integer ranging from 0 to 20.

4. The nanoparticle of claim 1, wherein each aryl group independently, for each occurrence, comprises a hydroxy group, an electron-withdrawing group, or a combination thereof.

5. The nanoparticle of claim 1, wherein the plurality of polymers comprise poly(4-vinylcatechol-co-maleic anhydride)-block poly(4-vinylcatechol) (PCatMA-b-PCat) diblock copolymers, wherein each polymer comprises a poly(4-vinylcatechol-co-maleic anhydride) (PCatMA) block and a poly(4-vinylcatechol) (PCat) block.

6. The nanoparticle of claim 1, wherein each polymer of the plurality of polymers independently has a structure according to Formula IA, Formula IA′, or Formula IA″: 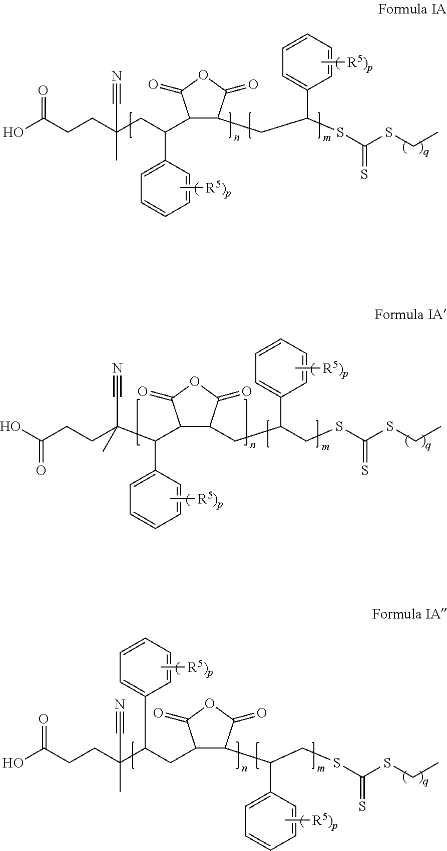
wherein each R⁵ independently, for each occurrence, is selected from —OH, a halogen, or a nitro group;
each p, for each occurrence, is an integer selected from 0 to 5; and
q is an integer selected from 0 to 20.

7. The nanoparticle of claim 1, wherein each polymer of the plurality of polymers Independently has a structure according to Formula IIB, IIB′, or IIB″: 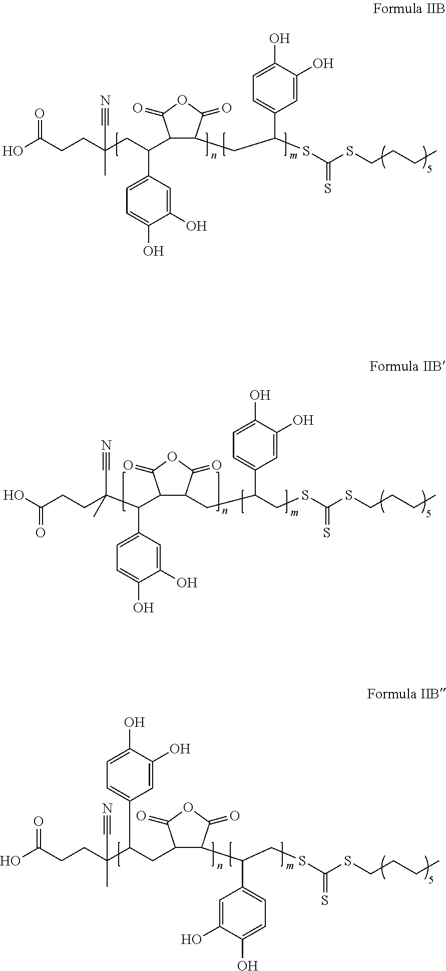
where m and n independently are integers ≥1.

8. The nanoparticle of claim 7, wherein:
m is an integer ranging from 10 to 500; and
n is an integer ranging from 10 to 500.

9. The nanoparticle of claim 1, wherein the polymers have a number average molecular weight ranging from 10 kDa to 150 kDa, as determined by gel permeation chromatography or nuclear magnetic resonance spectroscopy calculation.

10. The nanoparticle of claim 1, wherein a plurality of the nanoparticles has an average diameter of from 80 nm to 180 nm, as determined by dynamic light scattering.

11. The nanoparticle of claim 1, wherein the polymers have a size distribution with a polydispersity index of from 1.0 to 1.6, as determined by dynamic light scattering.

12. The nanoparticle of claim 1, wherein the nanoparticle has a micellar structure defining a core and wherein a therapeutic agent, a detectable label, or a combination thereof, is encapsulated in the core.

13. A pharmaceutical composition comprising:
a nanoparticle according to claim 1; and
at least one pharmaceutically acceptable excipient.

14. A method of synthesizing a nanoparticle, the method comprising:
preparing a plurality of polymers;
preparing a solution of the plurality of polymers in acetone; and
dialyzing the solution against water, whereby polymers of the plurality of polymers form into nanoparticles,
wherein each polymer of the plurality of polymers has a structure according to Formula I 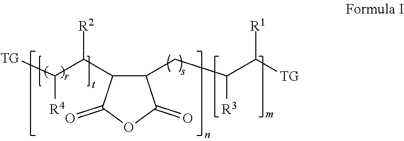
where m, n, and t, independently are integers ≥1;
each TG independently is a terminating group;
each R¹, R², R³, and R⁴, independently, for each occurrence, is H or comprises an aryl group;
each r independently, for each occurrence, is 1 or 0;
each s independently, for each occurrence, is 1 or 0; and
provided that,
(i) at least one R¹, R², R³, or R⁴, is not, or is other than, phenyl,
(ii) one of R¹ or R³, for each occurrence, comprises the aryl group, and
(iii) one of R² or R⁴, for each occurrence, comprises the aryl group.

15. The method of claim 14, wherein preparing the plurality of polymers comprises:
combining maleic anhydride and a vinyl aryl-containing monomer with a chain transfer agent, an initiator, and a solvent to form a monomer solution; and
heating the monomer solution at a temperature effective to induce polymerization and produce the plurality of polymers.

16. The method of claim 14, further comprising:
adding a therapeutic agent, a detectable label, or a combination thereof to the solution; and
wherein dialyzing the solution against water provides nanoparticles encapsulating the therapeutic agent, the detectable label, or the combination thereof.

17. A method, comprising administering the nanoparticle of claim 1, or a pharmaceutically acceptable composition thereof, to a subject having, or at risk of having, inflammation and/or iron-dependent cell death.

18. The method of claim 17, further comprising contacting a bone cell with the nanoparticle.

19. The method of claim 18, wherein the subject has a bone fracture and the bone cell is at a site of the bone fracture.

20. The method of claim 17, wherein the nanoparticle scavenges reactive oxygen species and/or chelates iron.
